(19) United States
(12) Patent Application Publication
(10) Pub. No.: US 2026/0272508 A1
(43) Pub. Date: Sep. 17, 2026

(54) DUAL SHAFT DELIVERY DEVICE

(71) Applicant: Medtronic, Inc., Minneapolis, MN (US)

(72) Inventors: Ronald A. Drake, St. Louis Park, MN (US); Lester O. Stener, Hudson, WI (US); Noah D. Barka, Ramsey, MN (US); Lindsey T Dahlberg, St Anthony, MN (US)

(21) Appl. No.: 19/473,402

(22) PCT Filed: Apr. 12, 2024
(86) PCT No.: PCT/US2024/024413

§ 371 Date: Oct. 7, 2025

Related U.S. Application Data
(60) Provisional application No. 63/496,168, filed on Apr. 14, 2023.
(60) Provisional application No. 63/593,142, filed on Oct. 25, 2023.

(51) Int. Cl. A61B 17/34; A61B 17/00

(52) U.S. Cl. CPC A61B 17/3468; A61B 2017/00243; A61B 2017/00292; A61B 2017/00367

(57) ABSTRACT

A delivery device (40) includes a first handle portion and a second handle portion. The first handle portion includes an inner shaft deflection mechanism (44). An inner shaft (46) is coupled to the first handle portion. The inner shaft is configured to deflect in a first deflection plane in response to actuation of the inner shaft deflection mechanism. The second handle portion includes an outer shaft deflection mechanism. The second handle portion is configured to rotate relative to the first handle portion. An outer shaft is coupled to the second handle portion. The outer shaft surrounds at least a portion of the inner shaft. The outer shaft is configured to deflect in a second deflection plane in response to actuation of the outer shaft deflection mechanism. Rotation of the second handle portion relative to the first handle portion causes the second deflection plane to rotate relative to the first deflection plane.

This application claims the benefit of U.S. Provisional Application Ser. No. 63/593,142, filed Oct. 25, 2023, and U.S. Provisional Application Ser. No. 63/496,168, filed Apr. 14, 2023, the entire content of each which is incorporated herein by reference.

### TECHNICAL FIELD

This disclosure relates to interventional medical systems and methods for delivering medical devices, such as to implant sites within a patient.

### BACKGROUND

Some types of implantable medical device systems may be configured to monitor one or more physiological parameters of a patient. Such systems may include one or more sensors that detect signals associated with physiological parameters of a patient. These implantable medical devices (IMDs) may be implanted subcutaneously. IMDs may allow clinicians to obtain patient data without the patient being connected to an external machine and/or present in a clinic. An IMD that is configured to continuously record one or more physiological parameters allows clinicians to review data over a longer period of time as compared with systems that use external monitoring equipment in a simulated testing situation.

### SUMMARY

In some examples, a delivery device comprises: a first handle portion comprising an inner shaft deflection mechanism; an inner shaft coupled to the first handle portion, wherein the inner shaft defines a longitudinally extending lumen, wherein the inner shaft mechanically supports a distal receptacle configured to receive an implantable medical device, and wherein the inner shaft is configured to deflect in a first deflection plane in response to actuation of the inner shaft deflection mechanism; a second handle portion comprising an outer shaft deflection mechanism, wherein the second handle portion is configured to rotate relative to the first handle portion; and an outer shaft coupled to the second handle portion, wherein the outer shaft surrounds at least a portion of the inner shaft; wherein the outer shaft is configured to deflect in a second deflection plane in response to actuation of the outer shaft deflection mechanism, wherein rotation of the second handle portion relative to the first handle portion causes the second deflection plane to rotate relative to the first deflection plane.

In some examples, a system comprises: an implantable medical device; and a delivery device comprising: a first handle portion comprising an inner shaft deflection mechanism; an inner shaft coupled to the first handle portion, wherein the inner shaft defines a longitudinally extending lumen, wherein the inner shaft mechanically supports a distal receptacle configured to receive an implantable medical device, and wherein the inner shaft is configured to deflect in a first deflection plane in response to actuation of the inner shaft deflection mechanism; a second handle portion comprising an outer shaft deflection mechanism, wherein the second handle portion is configured to rotate relative to the first handle portion; and an outer shaft coupled to the second handle portion, wherein the outer shaft surrounds at least a portion of the inner shaft; wherein the outer shaft is configured to deflect in a second deflection plane in response to actuation of the outer shaft deflection mechanism, wherein rotation of the second handle portion relative to the first handle portion causes the second deflection plane to rotate relative to the first deflection plane.

In some examples, a method comprises: inserting a distal receptacle of a delivery device into an access site on a body of a patient, wherein the distal receptacle is carrying an implantable medical device; navigating the delivery device toward an implant site; actuating an inner shaft deflection mechanism of the delivery device to cause an inner shaft of the delivery device to deflect in a first deflection plane; rotating a second handle portion of the delivery device relative to a first handle portion of the delivery device; actuating an outer shaft deflection mechanism of the delivery device to cause an outer shaft of the delivery device to deflect in a second deflection plane, wherein an angle between the first deflection plane and the second deflection plane is based on an amount of rotation of the second handle portion relative to the first handle portion; and securing the implantable medical device to the implant site.

The details of one or more examples are set forth in the accompanying drawings and the description below. Other features, objects, and advantages will be apparent from the description and drawings, and from the claims.

### BRIEF DESCRIPTION OF THE DRAWINGS

FIG. 1 is a conceptual diagram illustrating potential cardiac implant sites for an IMD.

FIG. 2 is a conceptual diagram illustrating an example IMD.

FIG. 3 is a conceptual diagram illustrating an example delivery device configured to deliver an IMD in accordance with techniques of this disclosure.

FIGS. 4A-4C are conceptual diagrams illustrating example usages of a delivery device in accordance with techniques of this disclosure.

FIG. 5 is a conceptual diagram illustrating a three-dimensional curvature of a delivery device in accordance with techniques of this disclosure.

FIG. 6 is a flow diagram illustrating an example technique for operating a delivery device in accordance with techniques of this disclosure.

FIGS. 7A-7G are conceptual diagrams of an example technique for operating a delivery device in accordance with techniques of this disclosure.

### DETAILED DESCRIPTION

An implantable medical device (IMD) may be configured to diagnose, monitor, and treat medical conditions. For example, a cardiac pacemaker may be configured to regulate a heart's rhythm, especially in cases of abnormal heart rates or rhythms. In general, an IMD may be delivered to various implant sites within the body of a patient. One example location is the Triangle of Koch (TOK), an anatomical landmark located in the right atrium of the heart that is proximate to the atrioventricular (AV) node and the His bundle.

Accurately implanting an IMD may be crucial for the IMD to function correctly. However, navigating through the venous system and positioning the IMD in the appropriate location can be challenging. For example, individual differences in anatomy, such as variations in heart size, shape, etc., can make it more difficult to deliver and place the IMD accurately in the heart. Additionally, the implant site (e.g., a specific region of the TOK) may not only be a relatively small area (e.g., compared to the size of the IMD), but also be moving (e.g., due to the heart beating), further increasing the difficulty of implantation. For example, implanting the IMD along the proper trajectory (e.g., for advancement of electrodes and/or fixation structures into tissue) may pose considerable challenges.

The present disclosure describes systems and techniques for delivering an IMD. For example, a device may include a first handle portion and a second handle portion. An inner shaft may be coupled to the first handle portion, and an outer shaft may be coupled to the second handle portion. The first handle portion may include an inner shaft deflection mechanism that causes the inner shaft to deflect from an initial configuration to a deflected configuration in a first deflection plane. Similarly, the second handle portion may include an outer shaft deflection mechanism that causes the outer shaft to deflect from an initial configuration to a deflected configuration in a second deflection plane. The second handle portion may be configured to rotate relative to the first handle portion (in turn rotating the outer shaft relative to the inner shaft), and rotation of the second handle portion relative to the first handle portion may rotate the second deflection plane relative to the first deflection plane. By performing some combination of rotating the second handle portion relative to the first handle portion, controlling the inner shaft deflection mechanism, and controlling the outer shaft deflection mechanism, a clinician may precisely manipulate the shape (e.g., the curvature) of the device in three-dimensional space. In this way, the device may be configured to better navigate (e.g., reach the target site) and orient (e.g., aim with the proper trajectory) with respect to an implant site, potentially decreasing the duration of the implant procedure and improving the likelihood of a successful implantation.

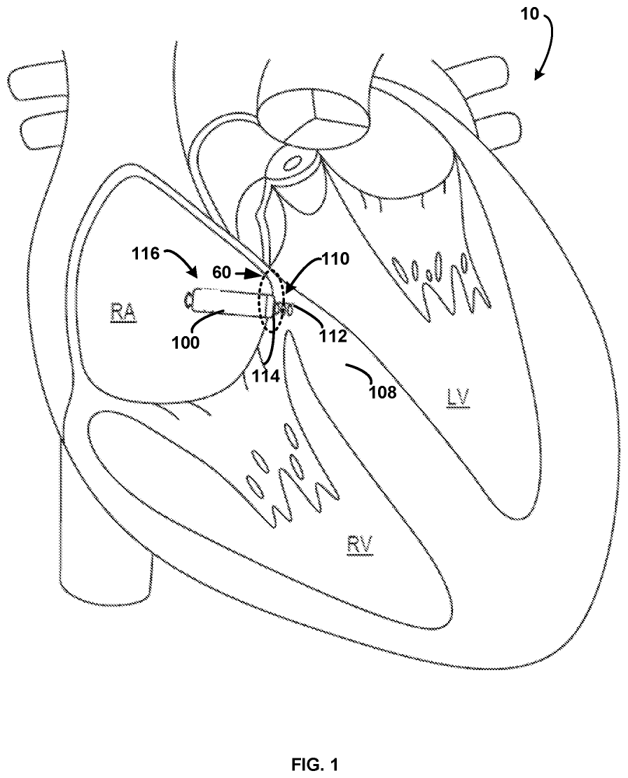

FIG. 1 is a conceptual diagram illustrating an example IMD 100 implanted in the heart 10 of a patient, in accordance with one or more aspects of this disclosure. IMD 100 is shown implanted in the right atrium (RA) of heart 10 in a target implant site 60, such as the triangle of Koch (TOK), in heart 10 of the patient with a distal end of IMD 100 directed toward the left ventricle (LV) of the patient's heart 10. Although in the example of FIG. 1 the distal end of IMD 100 is directed toward the LV, the distal end may be directed to other targets, such as the interventricular septum of heart 10. Target implant site 60 may lie between the His bundle and the coronary sinus and may be adjacent to the tricuspid valve.

IMD 100 includes a distal end 110 and a proximal end 116. Distal end 110 includes a first electrode 112, and a second electrode 114. First electrode 112 may define a helical shape, e.g., as illustrated in FIG. 1. First electrode 112 extends from distal end 110 and may penetrate through the wall tissue of a first chamber (e.g., the RA in the illustrated example) into wall tissue of a second chamber (e.g., ventricular myocardium 108 of the LV in the illustrated example). Second electrode 114 may be disposed on a ramp extending distally from distal end 110 and is configured to be placed in contact with the wall tissue of the first chamber without penetration of the wall tissue of the first chamber by second electrode 114. Second electrode 114 may contact the wall tissue of the first chamber as first electrode 112 penetrates the wall tissue of the first chamber.

The configuration of electrodes 112 and 114 illustrated in FIG. 1 allows IMD 100 to sense cardiac signals and/or deliver cardiac pacing to multiple chambers of heart 10, e.g., the RA and ventricle(s) in the illustrated example. In this manner, the configuration of electrodes 112 and 114 may facilitate the delivery of A-V synchronous pacing by a single IMD (e.g., IMD 100) implanted within the single chamber, e.g., the RA. While IMD 100 is implanted at implant site 60 to sense in and/or pace the RA and ventricle(s) in the example shown in FIG. 1, a device having an electrode configuration in accordance with the examples of this disclosure may be implanted at any of a variety of locations to sense in and/or pace any one, two, or more chambers of heart 10. For example, IMD 100 may be implanted at implant site 60 or another region, and first electrode 112 may extend into tissue, e.g., myocardial tissue, of the LV or interventricular septum to, for example, facilitate the delivery of A-V synchronous pacing. Furthermore, a device having an electrode configuration in accordance with the examples of this disclosure may be implanted at any of a variety of locations within a patient for sensing and/or delivery of therapy to other patient tissue. In some examples first electrode 112 may extend into the tissue of heart 10 at implant site 60 and affix IMD 100 to the tissue of heart 10. Consequently, IMD 100 may be employed to deliver pacing therapy to heart in any one or combination of, or all of the following pacing modes: AAI, VVI, DDD, AAI(R), VVI(R), DDD(R).

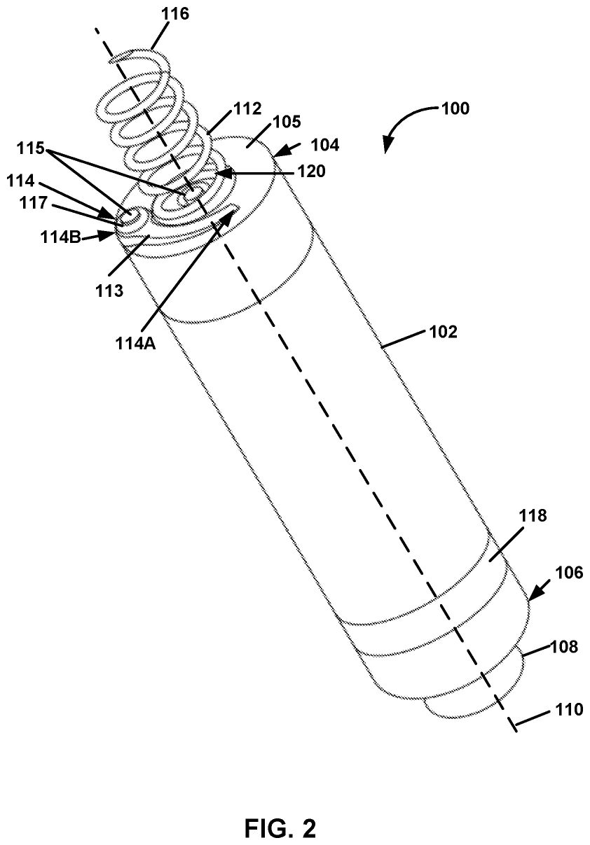

FIG. 2 is a perspective diagram illustrating IMD 100. IMD 100 includes a housing 102 that defines a hermetically sealed internal cavity. Housing 102 may be formed from a conductive material including titanium or titanium alloy, stainless steel, MP35N (a non-magnetic nickel-cobalt-chromium-molybdenum alloy), platinum alloy or other bio-compatible metal or metal alloy, or other suitable conductive material. In some examples, housing 102 is formed from a non-conductive material including ceramic, glass, sapphire, silicone, polyurethane, epoxy, acetyl co-polymer plastics, polyether ether ketone (PEEK), a liquid crystal polymer, other biocompatible polymer, or other suitable non-conductive material.

Housing 102 extends between a distal end 104 and a proximal end 106 along longitudinal axis 109. In some examples, housing can be cylindrical or substantially cylindrical but may be other shapes, e.g., prismatic, or other geometric shapes. Housing 102 may include a delivery tool interface member 107, e.g., at proximal end 106, for engaging with a delivery tool during implantation of IMD 100. At distal end 104, housing 102 may define a face 105 of housing 102. Face 105 may define a distal end major surface. Face 105 may be orthogonal to longitudinal axis 109. In some examples, face 105 may be slanted. For example, face 105 may define a reference plane that is not orthogonal to longitudinal axis 109.

IMD 100 may include a ramp 113. Ramp 113 may be configured to promote contact between an electrode (e.g., second electrode 114 described below) and wall tissue of the chamber of heart 10 without penetration of the wall tissue of the chamber by the electrode. Ramp 113 may further be configured to separate the electrode from distal end 104 by a fixed distance.

Ramp 113 may extend from a first end 119A that is fixedly attached to housing 102 at or near distal end 104 (e.g., attached to face 105), to a second end 119B that is more distal to first end 119A. Ramp 113 may be disposed radially outwards of first electrode 112 relative to longitudinal axis 109. Ramp 113 may extend around at least a portion of a perimeter of housing 102. Ramp 113 may extend up to 180 degrees around longitudinal axis 109 and along the perimeter of housing 102. Ramp 113 may be integrally formed as a part of the manufacturing of at least a portion of housing 102 (e.g., as a part of the manufacturing of a header defining distal end 104 and face 105 of housing 102). Ramp 113 may be formed via a molding process, via additive manufacturing, or the like. In some examples ramp 113 is formed separately and affixed to face 105 of housing 102 afterwards. Ramp 113 may define a partial helix, e.g., wound in a same direction and/or in different directions as a helix and/or coil defined by first electrode 112. Ramp 113 may be formed at least partially of an electrically conductive material, such as titanium, platinum, iridium, tantalum, or alloys thereof, and/or of electrically nonconductive material(s). At least portions of ramp 113 may be coated with an electrically insulating coating, e.g., a parylene, polyurethane, silicone, epoxy, or other insulating coating.

First electrode 112 may include one or more coatings (e.g., electrically insulative coating(s)) configured to define a first electrically active region 121, or first electrode 112 may otherwise define first electrically active region 121. In some examples, first electrically active region 121 may be more proximate to the second, e.g., distal, end of first electrode 112. In the example of FIG. 2, first electrically active region 121 includes the distal end of electrode 112. Second electrode 114 may include one or more coatings configured to define a second electrically active region 123 on an outer surface of electrode 114. In some examples, as illustrated in FIG. 2, second electrical active region 123 forms a ring around a therapeutic substance dispensing device 115. Second electrode 114 may include, but is not limited to, a button electrode, a spring electrode, or any other suitable type or shape of electrode.

First and second electrodes 112 and 114 may be formed of an electrically conductive material, such as titanium, platinum, iridium, tantalum, stainless steel or alloys thereof. First and second electrodes 112 and 114 may be coated with an electrically insulating coating, e.g., a parylene, polyurethane, silicone, epoxy, or other insulating coating, to reduce the electrically conductive active surface area of first and second electrodes 112 and 114, and thereby define first and second electrically active regions 121 and 123. Defining first and second electrically active regions 121 and 123 by covering portions with an insulating coating may increase the electrical impedance of first and second electrodes 112 and 114 and thereby reduce the current delivered during a pacing pulse that captures the cardiac tissue. A lower current drain conserves the power source, e.g., one or more rechargeable or non-rechargeable batteries, of IMD 100.

In some examples, first and second electrodes 112 and 114 may have an electrically conducting material coating on first and second electrically active regions 121 and 123 to define the active regions. For example, first and second electrically active regions 121 and 123 may be coated with titanium nitride (TiN). First and second electrodes 112 and 114 may be made of substantially similar material or may be made of different material from one another.

In the example of FIG. 2, first electrode 112 takes the form of a helix or a coil. First electrode 112 may be an elongated body defining a helix. In some examples, a helix is an object having a three-dimensional shape like that of a wire wound uniformly in a single layer around a cylindrical or conical surface or mandrel such that the wire would be in a straight line if the surface were unrolled into a plane. First electrode 112 may extend from face 105 from proximal end 120 to a distal end, e.g., defining first electrically active region 121. Proximal end 120 may be a location along first electrode 112 where first electrode 112 extends distally past distal end 104 of IMD 100.

Second electrode 114 is disposed on distal end 104 and may include a button electrode, e.g., as illustrated in FIG. 2, or any other suitable type or shape of electrode. In some examples, IMD 100 may have a plurality of second electrodes 114 (e.g., two or more second electrodes 114) disposed on distal end 104 of housing 102. The plurality of second electrodes 114 may be equally spaced around a circumference of distal end 104. At least one of the plurality of second electrodes 114 may be disposed on ramps (e.g., on two or more ramps 112). In some examples, each of the plurality of second electrodes 114 may be disposed on ramps. Each ramp 113 may include a single second electrode 114 or two or more second electrodes 114. In some examples, second electrode 114 may be disposed at a predetermined angle away from first end of first electrode 112.

First and second electrodes 112 and 114 may vary in size and shape in order to enhance tissue contact of first and second electrically active regions 121 and 123. For example, first electrodes 112 may have a round cross-section or could be made with a flatter cross-section (e.g., oval or rectangular) based on tissue contact specifications. In some examples, second electrode 114 may have an outer surface that varies in size and shape (e.g., an oval outer surface, an outer surface with a larger diameter, or the like) in order to enhance tissue contact of second electrically active region 123.

The size and shape of first electrode 112 may be determined at least in part by stiffness requirements. For example, stiffness requirements may vary based on the expected implantation requirements, including the tissue into which the electrodes are implanted or contact, as well as how long IMD 100 is intended to be implanted.

First electrode 112 may extend from housing distal end 104 approximately 3 mm to 12 mm in various examples. In some examples, first electrode 112 may extend a distance from distal end 104 by at least 3 mm, at least 3 mm but less than 20 mm, less than 15 mm, less than 10 mm, or less than 8 mm in various examples. The diameter of an elongated body defining first electrode 112 may be 2 mm or less, e.g., may be 1 mm or less, may be 0.6 mm or less. An outer diameter of the helix or coil defined by first electrode 112 may be 4 mm or less.

The distal end of first electrode 112 can have a conical, hemi-spherical, or slanted edge distal tip with a narrow tip diameter, e.g., less than 1 millimeter (mm), for penetrating into and through tissue layers. In some examples, the distal end of first electrode can be a sharpened or angular tip or sharpened or beveled edges, but the degree of sharpness may be constrained to avoid a cutting action that could lead to lateral displacement of the distal end of first electrode 112 and undesired tissue trauma. In some examples, first electrode 112 may have a maximum diameter at its base that interfaces with housing distal end 104. In such examples, the outer diameter of the helix defined by first electrode 112 may decrease from housing distal end 104 to the distal end of first electrode 112. In some examples, the diameter of first electrode 112 may vary from housing distal end 104 to the distal end of first electrode 112. The varying diameter may cause first electrode 112 to resist rotation within the tissue of heart 10.

The outer dimensions of first electrode 112 can be substantially straight and cylindrical, with first electrode 112 being rigid in some examples. In some examples, first electrode 112 may have flexibility in lateral directions, being non-rigid to allow some flexing with heart motion. In a relaxed state, when not subjected to any external forces, first electrode 112 can be configured to maintain a distance between first electrically active region 121 and housing distal end 104.

Distal end of first electrode 112 can pierce through one or more tissue layers to position first electrically active region 121 within a desired tissue layer, e.g., the ventricular myocardium 108 or interventricular septum. Accordingly, first electrode 112 extends a distance from housing distal end 104 corresponding to the expected pacing site depth and may have a relatively high compressive strength along its longitudinal axis, which may be substantially similar to or coincident with longitudinal axis 109, to resist bending in a lateral or radial direction when a longitudinal, axial, and/or rotational force is applied, e.g., to the proximal end 106 of housing 102 to advance IMD 100 into the tissue at implant site 60. By resisting bending in a lateral or radial direction, first electrode 112 can maintain a spacing between a plurality of windings of first electrode 112 when first electrode 112 is a helix electrode. The spacing may be a pre-determined pitch of first electrode 112 and may vary from distal end 104 to the distal end of first electrode 112. First electrode 112 may be longitudinally non-compressible. First electrode 112 may also be elastically deformable in lateral or radial directions when subjected to lateral or radial forces, however, to allow temporary flexing, e.g., with tissue motion, but returns to its normally straight position when lateral forces diminish. In some examples, when first electrode 112 is not exposed to any external force, or to only a force along its longitudinal axis (substantially similar to or coincident with longitudinal axis 109), first electrode 112 retains a straight, linear configuration as shown.

All, substantially all, or a portion of housing 102 may function as an electrode 118, e.g., an anode, during pacing and/or sensing. In some examples, electrode 118 can circumscribe a portion of housing 102 at or near proximal end 106. Electrode 118 can fully or partially circumscribe housing 102. FIG. 2 shows electrode 118 extending as a singular band. Electrode 118 can also include multiple segments spaced a distance apart along a longitudinal axis 109 of housing 102 and/or around a perimeter of housing 102. In some examples, electrode 118 may be disposed on face 105 or on another ramp 113 disposed on face 105. For example, electrode 114 may be disposed on a first ramp 113 and electrode 118 may be disposed on a second ramp 113.

When housing 102 is formed from a conductive material, such as a titanium alloy, portions of housing 102 may be electrically insulated by a non-conductive material, such as a coating of parylene, polyurethane, silicone, epoxy or other biocompatible polymer, or other suitable material. For the portions of housing 102 without the non-conductive material, one or more discrete areas of housing 102 with conductive material can be exposed to define electrode 118.

When housing 102 is formed from a non-conductive material, such as a ceramic, glass or polymer material, an electrically-conductive coating or layer, such as a titanium, platinum, stainless steel, alloys thereof, a conductive material may be applied to one or more discrete areas of housing 102 to form electrode 118.

In some examples, electrode 118 may be a component, such as a ring electrode, that is mounted or assembled onto housing 102. Electrode 118 may be electrically coupled to internal circuitry of IMD 100 via electrically-conductive housing 102 or an electrical conductor when housing 102 is a non-conductive material. In some examples, electrode 118 is located proximate to proximal end 106 of housing 102 and can be referred to as a proximal housing-based electrode. Electrode 118 can also be located at other positions along housing 102, e.g., located proximately to distal end 104 or at other positions along longitudinal axis 109.

In some examples, second electrode 114 or electrode 118 may be paired with first electrode 112 for sensing ventricular signals and delivering ventricular pacing pulses. In some examples, second electrode 114 may be paired with electrode 118 or first electrode 112 for sensing atrial signals and delivering pacing pulses to atrial tissue (e.g., to the atrial myocardium) in implant site 60. In other words, electrode 118 may be paired, at different times, with first electrode 112 and/or second electrode 114 for either ventricular or atrial functionality, respectively, in some examples. In some examples, first and second electrodes 112 and 114 may be paired with each other, with different polarities, for atrial and ventricular functionality.

In some examples, second electrode 114 may be configured as an atrial cathode electrode for delivering pacing pulses to the atrial tissue, e.g., at implant site 60 in combination with electrode 118. Second electrode 114 and electrode 118 may also be used to sense atrial P-waves for use in controlling atrial pacing pulses (delivered in the absence of a sensed P-wave) and for controlling atrial-synchronized ventricular pacing pulses delivered using first electrode 112 as a cathode and electrode 118 as the return anode.

A distal end of first electrode 112 can be configured to rest within a ventricular myocardium of the patient, and second electrode 114 and ramp 113 can be configured to contact an atrial endocardium of the patient. IMD 100 may include more or fewer electrodes than two electrodes. In some examples, IMD 100 may include one or more second electrodes 114 along housing distal end 104. For example, IMD 100 may include two or three electrodes configured for atrial functionality like second electrode 114, and the three electrodes may be substantially similar or different from one another. Spacing between a plurality of second electrodes 114 may be at an equal or unequal distance. Second electrode(s) 114 may be individually selectively coupled to sensing and/or pacing circuitry enclosed by housing 102 for use as an anode with first electrode 112 or as an atrial cathode electrode, or may be electrically common and not individually selectable. In some examples, in place of first electrode 112, IMD 100 may include a fixation element (not shown) of similar shape and mechanical, but without an electrically active region or electrode formed thereon or borne thereby; in such examples, electrically active region 121 can be positioned on a separate member and/or on the housing 102.

In some examples IMD 100 includes one or more therapeutic substance dispensing devices 115, e.g., on face 105, within a recess defined by second electrode 114, on ramp 113. The therapeutic substance (e.g., a steroid) may mitigate inflammation of patient tissue resulting from interaction with the IMD. Therapeutic substance dispensing devices 115 may be configured to elute one or more steroids to tissue in proximity to therapeutic substance dispensing devices 115 over time. In some examples, therapeutic substance dispensing devices 115 comprise one or more monolithic controlled release devices (MCRDs).

In some examples, IMD 100 includes one or more therapeutic substance dispensing devices 115 configured to elute one or more steroids to tissue proximate to first electrode 112. Therapeutic substance dispensing devices 115 may be disposed within a recess defined by second electrode 114. In some examples, therapeutic substance dispensing devices 115 may be disposed at a center of face 105, e.g., within recess defined by housing 102, and/or on ramp 113, e.g., between first end 119A and second end 119B. In some examples, first electrode 112 may deliver a therapeutic substance. For example, first electrode 112 may be coated with the therapeutic substance or include therapeutic substance dispensing devices 115.

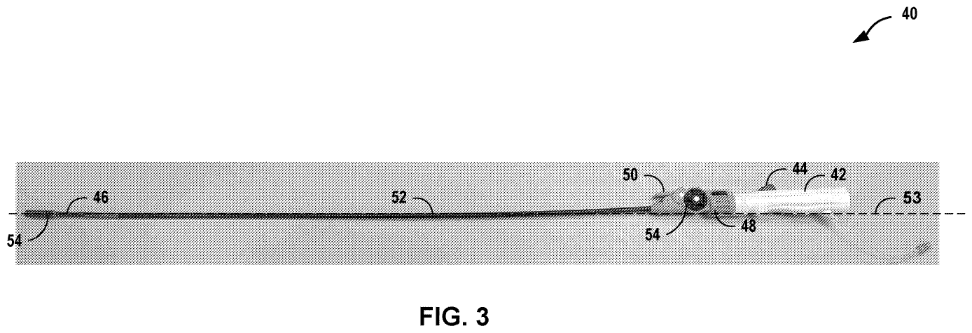

FIG. 3 is a conceptual diagram illustrating a dual shaft delivery device 40 (“delivery device 40”) configured to deliver IMD 100 in accordance with techniques of this disclosure. Delivery device 40 may include a first handle portion 42 that a clinician may grasp and manipulate during an implant procedure. First handle portion 42 may be shaped to be ergonomic and to facilitate precise control and ease of use. First handle portion 42 may include various components, such as knobs, dials, or other controls for adjusting the position, orientation, etc., of IMD 100 as IMD 100 is being placed within a patient's body. For example, first handle portion 42 may include an inner shaft deflection mechanism 44 that may be configured to cause the deflection of an inner shaft 46 of delivery device 40. Inner shaft 46 may define a longitudinally extending lumen.

Delivery device 40 may also include a second handle portion 48. Second handle portion 48 may be configured to rotate relative to first handle portion 42 (and, consequently, first handle portion 42 may be configured to rotate relative to second handle portion 48). Like first handle portion 42, second handle portion 48 may include various components, such as an outer shaft deflection mechanism 50. Outer shaft deflection mechanism 50 may be configured to cause the deflection of an outer shaft 52 of delivery device 40. Outer shaft 52 may surround at least a portion of inner shaft 46. For example, outer shaft 52 may define a lumen in which inner shaft 46 is positioned. In some examples, an inner diameter of outer shaft 52 and an outer diameter of inner shaft 46 may be sized to prevent fluid from flowing between outer shaft 52 and inner shaft 46. In this way, inner shaft 46 and outer shaft 52 may form a seal at a distal end of delivery device 40.

In some examples, first handle portion 42 and second handle portion 48 may be coupled together in an integral handle unit. First handle portion 42 may be disposed at a first longitudinal position on a longitudinal axis 53 of delivery device 40. Second handle portion 48 may be disposed at a second longitudinal position on longitudinal axis 53 of delivery device 40. The first longitudinal position may be different from the second longitudinal position. For example, the second longitudinal position may be distal to the first longitudinal position.

In some examples, second handle portion 48 and first handle portion 42 may be unitary in that second handle portion 48 cannot slide or otherwise translate along longitudinal axis 53 of delivery device relative to first handle portion 42. In these examples, delivery device 40 may be non-telescoping (e.g., a proximal end of inner shaft 46 may be unable to slide a significant amount relative to a proximal end of outer shaft 52). In other examples, second handle portion 48 may be able to slide or otherwise translate along longitudinal axis 53 of delivery device 40. In these examples, delivery device 40 may be telescoping (e.g., a proximal end of inner shaft 46 may be able to slide a significant amount relative to a proximal end of outer shaft 52).

Inner shaft 46 may be coupled to (e.g., extend from, connected to, etc.) first handle portion 42, and outer shaft 52 may be coupled to second handle portion 48. To facilitate navigating IMD 100 through blood vessels and other body structures, inner shaft 46 and outer shaft 52 may configured to be highly flexible and maneuverable. For example, inner shaft 46 and outer shaft 52 may be formed polyurethane, polyethylene, polyether block amide, PTFE (polytetrafluoroethylene), etc., which may provide flexibility, kink resistance, and structural integrity. In some examples, inner shaft 46 and/or outer shaft 52 may be multi-segmented or multi-layered structures to increase flexibility while maintaining strength. For example, inner shaft 46 and/or outer shaft may be reinforced with braided wires embedded within the sidewalls of inner shaft 46 and/or outer shaft 52 to improve the pushability and torque control of delivery device 40, allowing for better navigation.

Inner shaft 46 may mechanically support a distal receptacle 54. Distal receptacle 54 may be configured to receive an IMD, such as IMD 100. For example, distal receptacle 54 may include one or more walls defining a cavity having an inner diameter sized to contain an IMD therein. In some examples, distal receptacle 54 may be substantially straight (e.g., the walls of distal receptacle 54 may be substantially parallel to a longitudinal axis defined by inner shaft 46. Distal receptacle 54 may define a cup (e.g., is in the shape of a cup). In some examples, distal receptacle 54 may support an electrode (e.g., on the surface of distal receptacle 54), which may be used for receiving an electrogram from tissue (e.g., atrial tissue). A distal end of outer shaft 52 may be proximal to distal receptacle 54. Additionally, an inner diameter of outer shaft 52 may be smaller than an outer diameter of distal receptacle 54, such that outer shaft 52 cannot slip over distal receptacle 54.

A clinician may use first handle portion 42 to navigate and orient inner shaft 46 within vasculature of a patient. For example, inner shaft 46 may be configured to deflect in a first deflection plane in response to actuation of inner shaft deflection mechanism 44. In some examples, one or more pull wires may extend from inner shaft deflection mechanism 44 to a distal region of inner shaft 46. When an operator adjusts or otherwise manipulates inner shaft deflection mechanism 44, the pull wire may apply a tensile force to inner shaft 46, causing inner shaft 46 to curve and assume a corresponding deflected inner shaft configuration.

A clinician may use second handle portion 48 to navigate and orient outer shaft 52 within a patient's body. For example, outer shaft 52 may be configured to deflect in a second deflection plane in response to actuation of outer shaft deflection mechanism 50. In some examples, one or more pull wires may extend from outer shaft deflection mechanism 50 to a distal region of outer shaft 52. When an operator adjusts or otherwise manipulates outer shaft deflection mechanism 50, the pull wire may apply a tensile force to outer shaft 52 causing outer shaft 52 to curve and assume a corresponding deflected outer shaft configuration.

Additionally, the rotation of second handle portion 48 relative to first handle portion 42 may cause a second deflection plane of outer shaft 52 to rotate, e.g., about longitudinal axis 53, relative to a first deflection plane of inner shaft 46. In other words, an angle, e.g., having an origin at longitudinal axis 53, between the first deflection plane and the second deflection plane may be based on an amount of rotation of second handle portion 48 relative to first handle portion 42. In this way, the techniques of this disclosure may enable manipulation of the shape of delivery device 40 in three-dimensional space.

For example, as shown in FIG. 3, when delivery device has 40 has no curve actuated, outer shaft 52 and inner shaft 46 may share or occupy the same plane as longitudinal axis 53 (e.g., the longitudinal plane of delivery device 40). When inner shaft 46 is deflected, the distal portion of inner shaft 46 may curve but still occupy the longitudinal plane of delivery device 40. When inner shaft 46 is rotated, inner shaft 46 may no longer be in the longitudinal plane of delivery device 40 but still share a plane with longitudinal axis 53. In other words, the curve formed by inner shaft 46 may be flat.

When outer shaft 52 is deflected, the distal portion of outer shaft 52 may curve but still occupy the longitudinal plane of delivery device 40. When outer shaft 52 is rotated, outer shaft 52 may no longer be in the longitudinal plane of delivery device 40 but still share a plane with longitudinal axis 53. In other words, the curve formed by outer shaft 52 may be flat. When inner shaft 46 and outer shaft 52 are deflected at the same time and inner shaft 46 is rotated relative to outer shaft 52, the deflection plane of outer shaft 52 may include longitudinal axis 53, but the deflection plane of inner shaft 46 may not.

In the example of FIG. 3, inner shaft deflection mechanism 44 is illustrated as a slider (that may be actuated by, e.g., sliding inner shaft deflection mechanism 44), and outer shaft deflection mechanism 50 is illustrated as a lever (that may be actuated by e.g., rotating outer shaft deflection mechanism 50). However, other form factors for inner shaft deflection mechanism 44 and outer shaft deflection mechanism 50 are contemplated by this disclosure. For example, inner shaft deflection mechanism 44 and/or outer shaft deflection mechanism 50 may be sliders, dials, knobs, levers, etc. In any case, by adjusting inner shaft deflection mechanism 44 and/or outer shaft deflection mechanism 50, a clinician can precisely steer delivery device 40 to navigate through challenging anatomy and orient toward a target implant site.

Although outer shaft deflection mechanism 50 may be any suitable mechanism (e.g., have any suitable form factor), it may be advantageous for outer shaft deflection mechanism 50 to be a lever. For example, deflection of outer shaft 52 may cause corresponding deflection of inner shaft 46 because outer shaft 52 surrounds at least a portion of inner shaft 46. Deflecting outer shaft 52 and inner shaft 46 at the same time may require a greater amount of force. As such, the amplification of force provided by a lever (e.g., mechanical advantage) may allow a clinician to more comfortably and precisely control outer shaft deflection mechanism 50.

In some examples, inner shaft 46 and outer shaft 52 may be biased to return to an initial inner shaft configuration and an initial outer shaft configuration, respectively. For various reasons, it may be inconvenient or impractical for a clinician to resist the bias (particularly the bias of outer shaft 52) and manually maintain the deflected configuration of inner shaft 46 and outer shaft 52. Accordingly, delivery device 40 may include a deflection locking mechanism 54 configured to maintain an outer shaft configuration of outer shaft 52. In this way, deflection locking mechanism 54 may be configured to resist outer shaft 52 from returning to the initial outer shaft configuration when deflection locking mechanism 54 is actuated.

In some examples, deflection locking mechanism 54 may be configured to apply a friction force to the pull wire extending from outer shaft deflection mechanism 50 to outer shaft 52, thereby resisting translation of the pull wire. For example, deflection locking mechanism 54 may be a dial that, when rotated, clamps onto a portion of the outer shaft deflection mechanism 50, generating a corresponding friction force. The generated friction force may depend on the degree of actuation of deflection locking mechanism 54, allowing for multiple use cases. For example, a clinician may cause deflection locking mechanism 54 to tightly clamp such that deflection locking mechanism 54 needs to be loosened before outer shaft 52 may return to the initial outer shaft configuration or be further deflected.

Alternatively, a clinician may cause deflection locking mechanism 54 to clamp just tightly enough to overcome the bias of outer shaft 52 such that outer shaft 52 cannot return to the initial outer shaft configuration but a clinician may further deflect outer shaft 52 without loosening deflection locking mechanism 54.

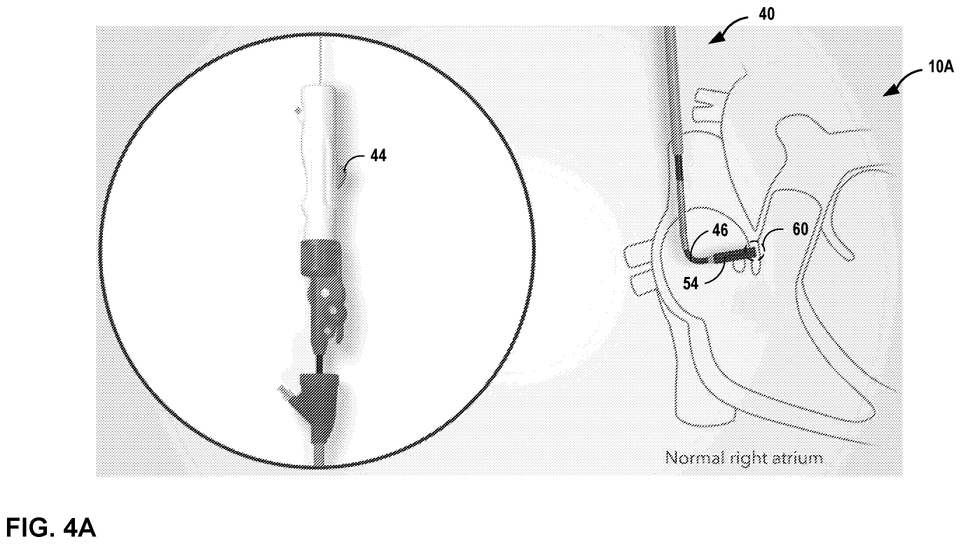

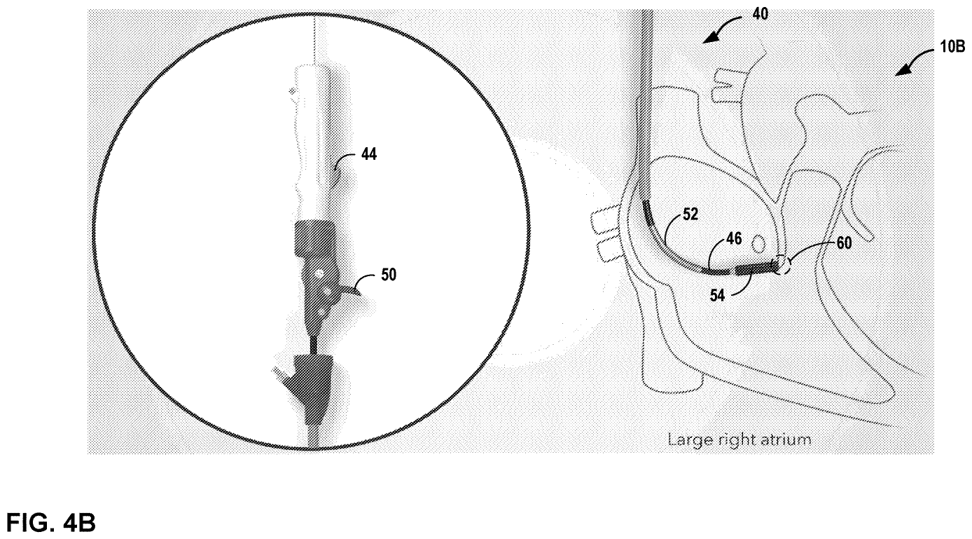

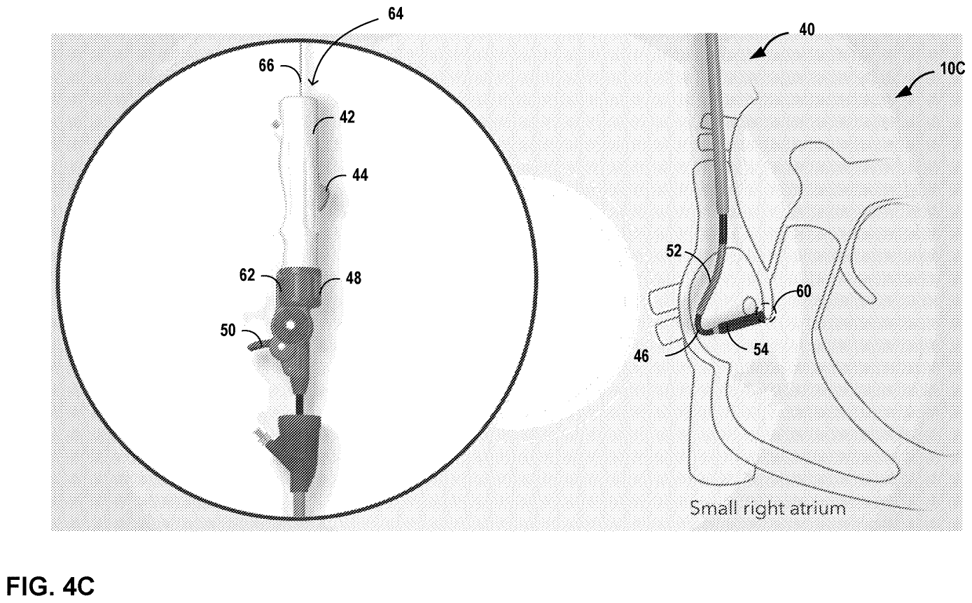

FIG. 4A is a conceptual diagram illustrating an example usage of delivery device 40. As shown in FIG. 4A, a clinician may navigate distal receptacle 54 of delivery device 40 to implant site 60 within a heart 10A, which may have a normal-sized right atrium. To facilitate navigation and orientation, the clinician may control various mechanisms of delivery device 40. For example, the clinician may actuate inner shaft deflection mechanism 44 (e.g., by sliding inner shaft deflection mechanism 44 in a first direction) to deflect inner shaft 46. Although illustrated in FIG. 4A as a deflection of approximately 90-degrees, a distal curve of inner shaft 46 may be any suitable deflection angle, such as a deflection between about 1-degree and about 180-degrees (e.g., between about 45-degree and 135-degrees). In any case, the distal curve of inner shaft 46 may have a relatively small radius of curvature such that delivery device 40 may orient distal receptacle 54 toward implant site 60 within the limited spaced provided by a normal-sized right atrium. In general, the deflection of inner shaft 46 in isolation may be in two-dimensional space.

FIG. 4B is a conceptual diagram illustrating an example usage of delivery device 40. As shown in FIG. 4B, a clinician may navigate distal receptacle 54 of delivery device 40 to implant site 60 within a heart 10B, which may have a relatively large right atrium. To facilitate navigation and orientation, the clinician may control various mechanisms of delivery device 40. For example, the clinician may actuate outer shaft deflection mechanism 50 to deflect outer shaft 52. Although illustrated in FIG. 4B as a deflection of approximately 90-degrees, a distal curve of outer shaft 52 may be any suitable deflection angle, such as a deflection between about 1-degree and about 180-degrees (e.g., between about 45-degree and 135-degrees). In any case, the overall distal curve of the outer shaft 52 and inner shaft 46 may have a relatively large radius of curvature such that delivery device 40 may orient distal receptacle 54 toward implant site 60 within a relatively large right atrium. In general, the deflection of outer shaft 46 in isolation may be in two-dimensional space.

FIG. 4C is a conceptual diagram illustrating an example usage of delivery device 40. As shown in FIG. 4C, a clinician may navigate distal receptacle 54 of delivery device 40 to implant site 60 within a heart 10C, which may have a relatively small right atrium. To facilitate navigation and orientation, the clinician may control various mechanisms of delivery device 40. For example, the clinician may actuate inner shaft deflection mechanism 44 to deflect inner shaft 46. The clinician may also actuate outer shaft deflection mechanism 50 to deflect outer shaft 52. In some examples, the clinician may rotate second handle portion 48 relative to first handle portion 42 (and in turn rotate outer shaft 52 relative to inner shaft 46) to provide more degrees of freedom for delivery device 40. In this way, delivery device 40 may better navigate and orient distal receptacle 54 toward implant site 60. For example, as shown in FIG. 4C, second handle portion 48 may be rotated about 180 degrees relative to first handle portion 42 such that actuation of outer shaft deflection mechanism 50 causes outer shaft 52 to deflect in a different direction relative to heart 10C (e.g., compared to the deflection of outer shaft 52 shown in FIG. 4B).

When second handle portion 48 has been rotated relative to first handle portion 42, a force may be required to maintain the rotational angle of second handle portion 48 relative to first handle portion 42. To address this issue, delivery device 40 may include a rotation locking mechanism 62 configured to maintain a rotational angle of second handle portion 48 relative to first handle portion 42. For example, when the clinician is satisfied with a rotational angle of second handle portion 48 relative to first handle portion 42, the clinician may actuate rotation locking mechanism 62 (e.g., by tightening rotation locking mechanism 62) to resist (e.g., using a friction force) the rotation of second handle portion 48.

Although illustrated in FIG. 4C as having been rotated about 180 degrees relative to first handle portion 42, second handle portion 48 may be rotated any amount (e.g., between about 1-degree and about 360-degrees to enable delivery device 40 to achieve a suitable curvature for navigating and orienting distal receptacle 54 toward implant site 60. In some examples, delivery device 40 may include visual indicia that indicate a degree of rotation of second handle portion 48 relative to first handle portion 42. By performing some combination of actuating inner shaft deflection mechanism 44, actuating outer shaft deflection mechanism 50, and rotating second handle portion 48 relative to first handle portion 42, a clinician may cause delivery device 40 to deflect in three-dimensional space.

In any case, once a clinician has navigated and oriented IMD 100 using delivery device 40, the clinician may implant IMD 100 to implant site 60. In some examples, the clinician may implant IMD 100 using the techniques and/or systems, tools, or assemblies as described in U.S. Pat. No. 11,331,475 , the entire content of which is incorporated herein by reference. For example, a lumen 64 of inner shaft 46 may be sized to receive a tether assembly 66. Tether assembly 66 may be configured to attach to IMD 100 such that rotation of tether assembly 66 causes a corresponding rotation of IMD 100. Thus, if IMD 100 is in contact with tissue at implant site 60, a clinician may rotate tether assembly 66 to screw first electrode 112 (e.g., which may be helically-shaped) of IMD 100 into the tissue, thereby securing IMD 100.

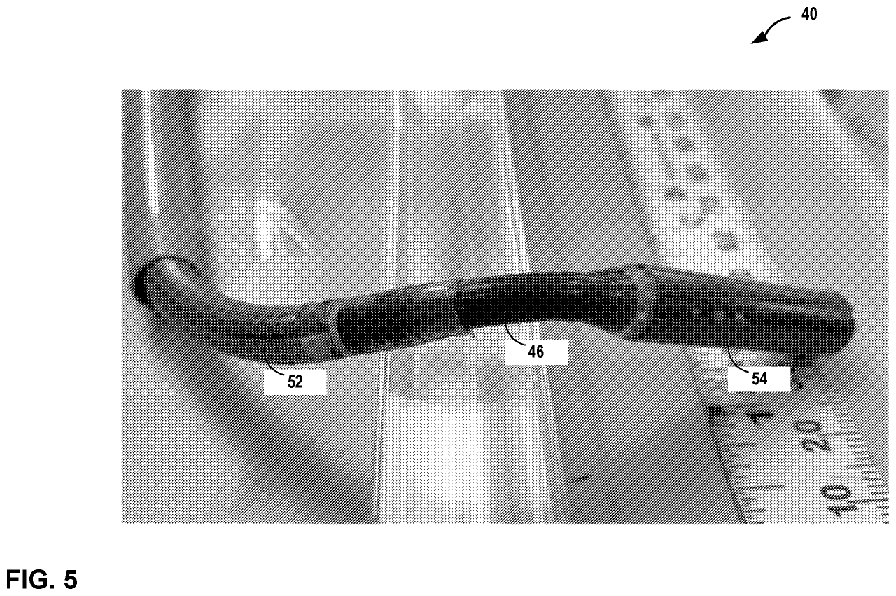

FIG. 5 is a conceptual diagram illustrating a three-dimensional curvature of delivery device 40. As shown in FIG. 5, a clinician may control various mechanisms of delivery device 40 to cause delivery device 40 to deflect in three-dimensional space. This ability to precisely and substantially manipulate the curvature of delivery device 40 may improve patient outcomes by, for example, reducing the time required to deliver IMD 100 to implant site 60 and the effort required to correctly navigate and orient IMD 100 with respect to implant site 60. The ability of delivery device 40 to effectively orient IMD 100 may be particularly useful when IMD 100 is a leadless device or when IMD 100 is not being implanted to ablate implant site 60.

Typical or existing intracardiac catheters are used for delivery of energy, fluid or devices to the cardiac tissue where orientation of a longitudinal axis defined by a distal end/portion of the catheter to the cardiac tissue is not as critical. The delivery device of the present invention provides significant advantages in effectiveness, and ease of orientation of the long axis of distal portion is important for procedure efficacy. For example, delivery device 40 may facilitate the successful implantation of IMD 100, which may require advancing first electrode 112 through the atrial myocardium and central fibrous body along a specific trajectory to position first electrically active region 121 in the ventricular myocardium. Such implantations are generally very challenging; however, the control and precision afforded by the delivery device of the present invention may substantially reduce the difficulty of such an operation. For example, delivery device 40 may facilitate better navigation (e.g., reach the target site) and orientation (e.g., aim with the proper trajectory).

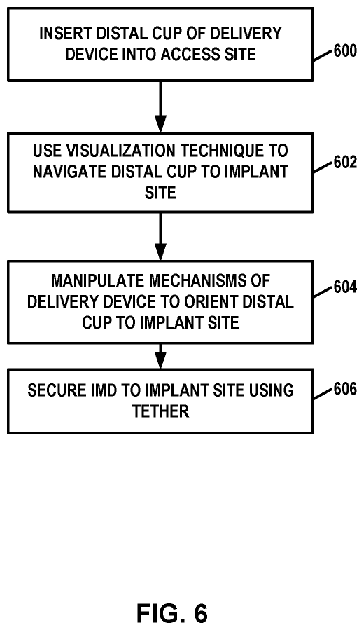

FIG. 6 is a flow diagram illustrating an example technique for operating delivery device 40 in accordance with techniques of this disclosure. Distal receptacle 54 of delivery device 40 may be inserted into an access site on a patient's body (600). An example access site may lead to vasculature of the patient, such as the internal jugular vein. A clinician may navigate delivery device 40 toward implant site 60, which may be (but is not necessarily) within heart 10. For example, the clinician may navigate delivery device 40 into the right atrium of heart 10, proximate the TOK.

During the implantation procedure, the clinician may use any appropriate visualization technique (602). For example, the implantation procedure may be performed under fluoroscopic guidance. Additionally or alternatively, a secondary marker may be placed in the coronary sinus to help cue the fluoroscopic view and movement of delivery device 40. For example, the secondary marker may act as a landmark for navigation because implant site 60 may be between the coronary sinus and tricuspid valve. A clinician may also use an electrogram, contrast, etc., in conjunction with the other visualization techniques discussed herein.

To properly position distal receptacle 54 (and in turn IMD 100) against implant site 60, the clinician may manipulate various mechanisms of delivery device 40 (604). For example, the clinician may actuate inner shaft deflection mechanism 44 to cause inner shaft 46 to deflect towards the ventricle. The clinician may rotate first handle portion 42 to orient distal receptacle 54 across the tricuspid valve and towards the septum. The clinician may withdraw delivery device 40 such that distal receptacle 54 is above yet adjacent to implant site 60. If necessary, the clinician may actuate outer shaft deflection mechanism 50 to deflect outer shaft 52 such that distal receptacle 54 is at this point oriented at the left ventricular apex and/or left ventricular free wall.

The clinician may control the orientation of distal receptacle 54 in three-dimensional space. For example, under fluoroscopy, a clinician may observe an outline of first electrode 112. Depending on a rotational angle of first electrode 112, first electrode 112 may appear as a series of loops or a sine wave. Thus, based on the orthogonal projection of first electrode 112 under fluoroscopy (or any other suitable visualization technique), the clinician may rotationally orient first electrode 112.

Once IMD 100 has not only been navigated to (e.g., positioned near) but also orientated towards (e.g., aimed at) implant site 60, the clinician may secure IMD 100 to implant site 60 (606). In some examples, the clinician may rotate a mechanical tether attached to IMD 100 until the clinician feels torsional feedback through the mechanical tether. The clinician may also count the number of revolutions of the mechanical tether.

First electrode 112 may be inserted such that tissue becomes engaged with the helix of first electrode 112. As first electrode 112 becomes engaged with tissue, first electrode 112 may pierce into the tissue at implant site 60 and advances through the atrial myocardium and central fibrous body of heart 10 to position first electrically active region 121 in the ventricular myocardium. In some examples, first electrode 112 penetrates into the interventricular septum, e.g., to the deep septum. In some examples, first electrode 112 does not perforate either of the ventricular endocardial or epicardial surface.

In some examples, manual pressure applied to the housing proximal end 106, e.g., via an advancement tool, provides the longitudinal force to pierce the cardiac tissue at implant site 60. In some examples, actuation of an advancement tool rotates device 104 and first electrode 112 configured as a helix about longitudinal axis 109. The rotation of the helix about the longitudinal axis 109 may advance first electrode 112 through the atrial myocardium and central fibrous body to position first electrically active region 121 in the ventricular myocardium.

Implant site 60 in some pacing applications is along the atrial endocardium, substantially inferior to the AV node and His bundle. First electrode 112 can have a length that penetrates through the atrial endocardium in implant site 60, through the central fibrous body and into the ventricular myocardium without perforating through the ventricular endocardial surface. In some examples, when the full length of first electrode 112 is fully advanced into implant site 60, first electrically active region 121 rests within the ventricular myocardium, and second electrode 114 is in contact with the atrial endocardium. In some examples, first electrode 112 and/or first electrically active region 121 is positioned in a location (e.g. the deep septum and/or LV septum) where IMD 100 drives physiologic contraction of the ventricles and/or provides physiologic pacing of the ventricles, e.g. via the conduction system of the heart and/or by activating the LV septum, employing first electrode 112 and/or first electrically active region 121. Instead of or in addition to such deep septal/LV septal and/or physiologic pacing, IMD 100 may be employed to deliver pacing therapy to heart in any one or combination of, or all of the following pacing modes: AAI, VVI, DDD, AAI(R), VVI(R), DDD(R).

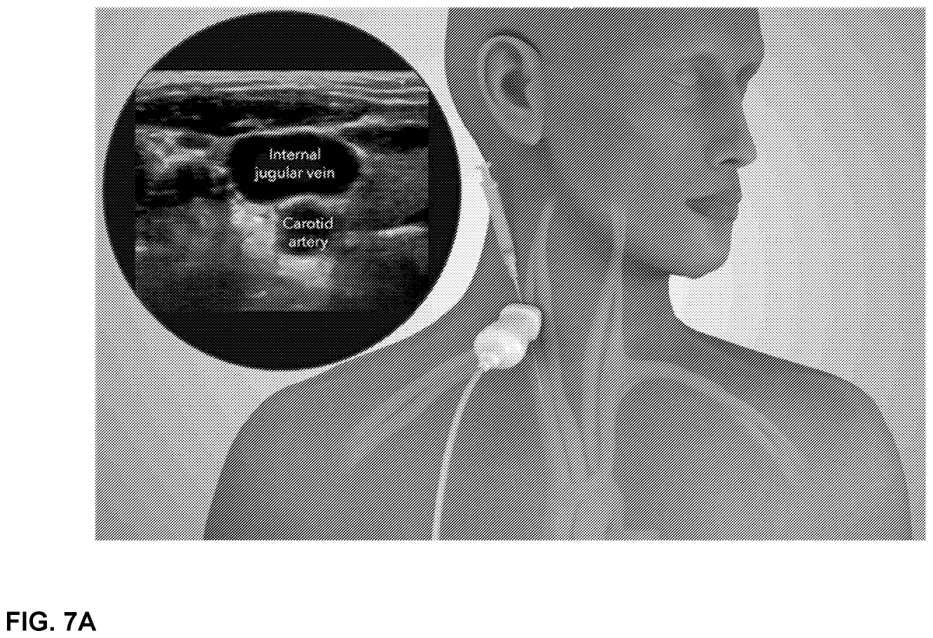

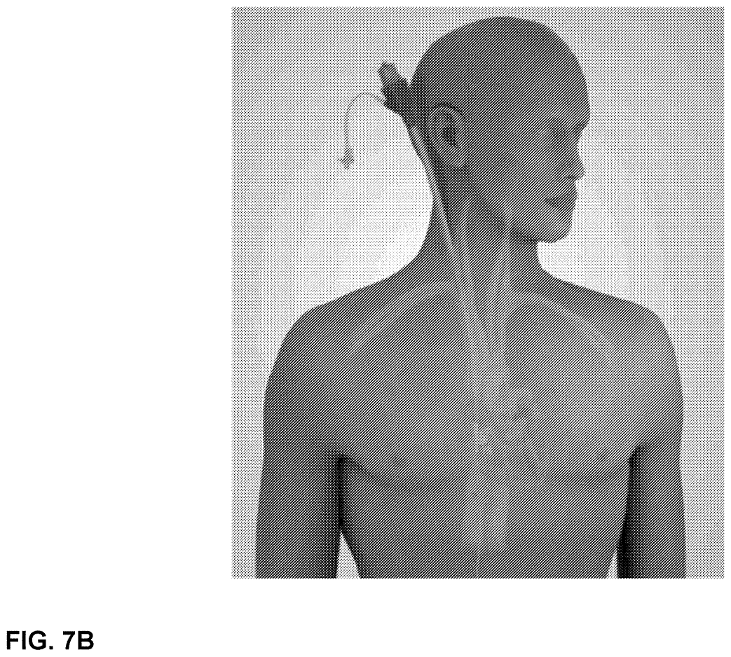

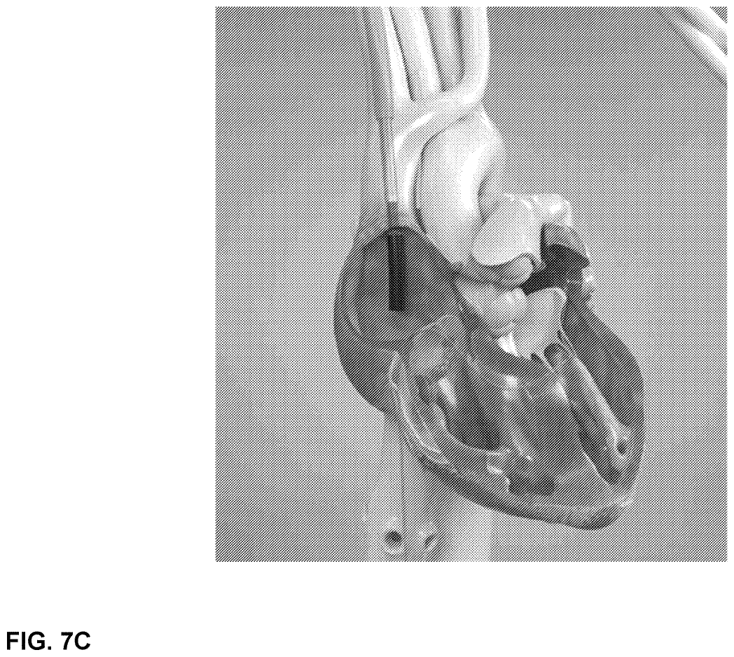

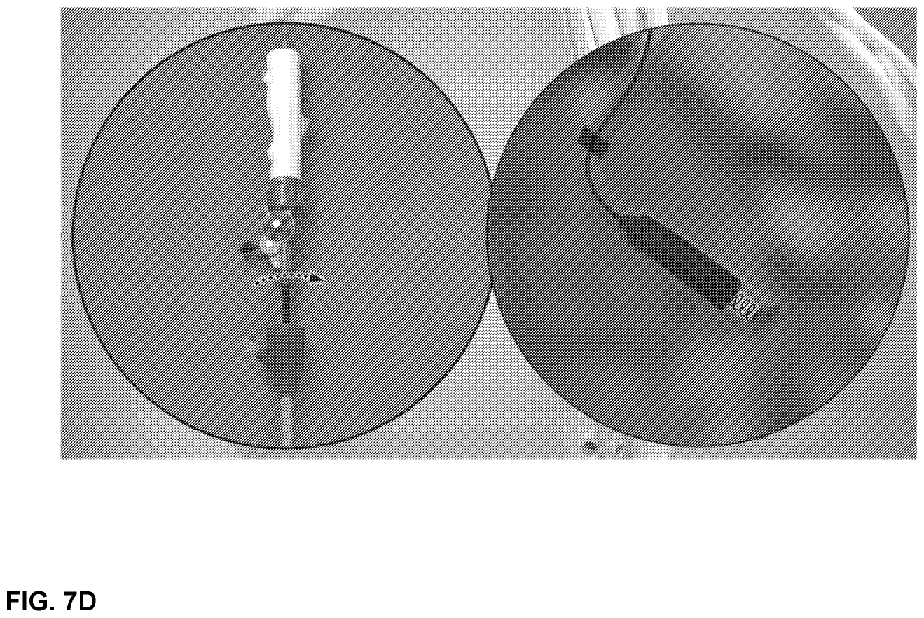

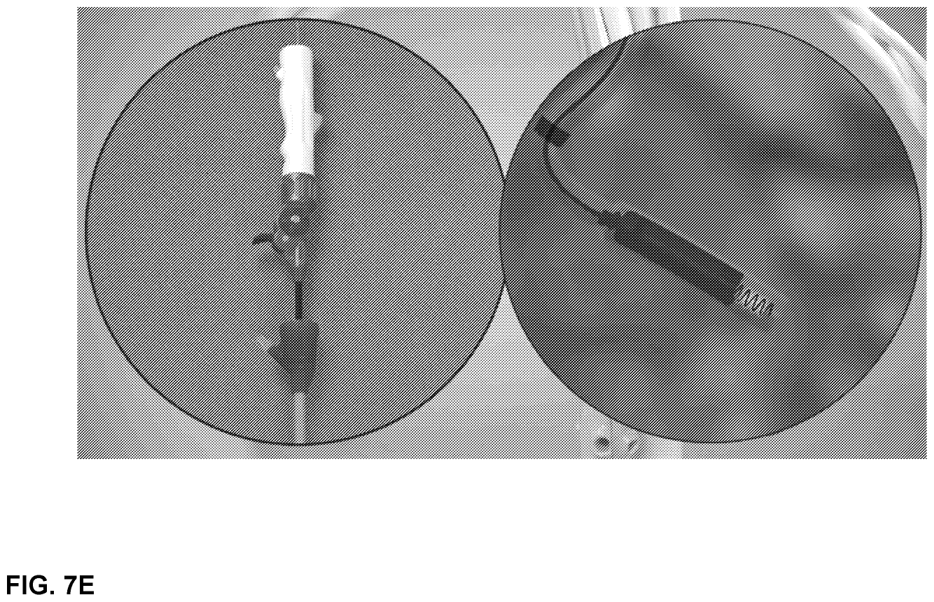

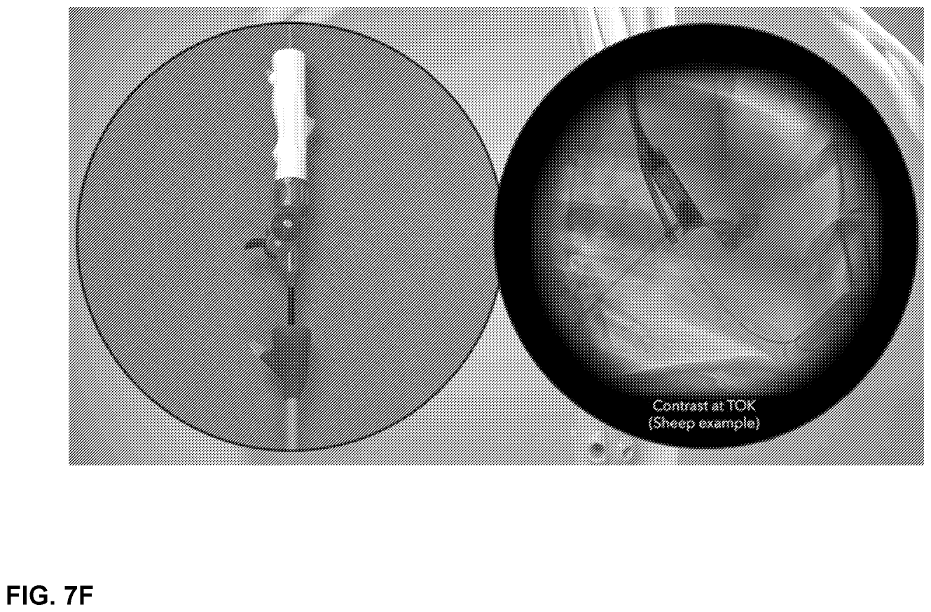

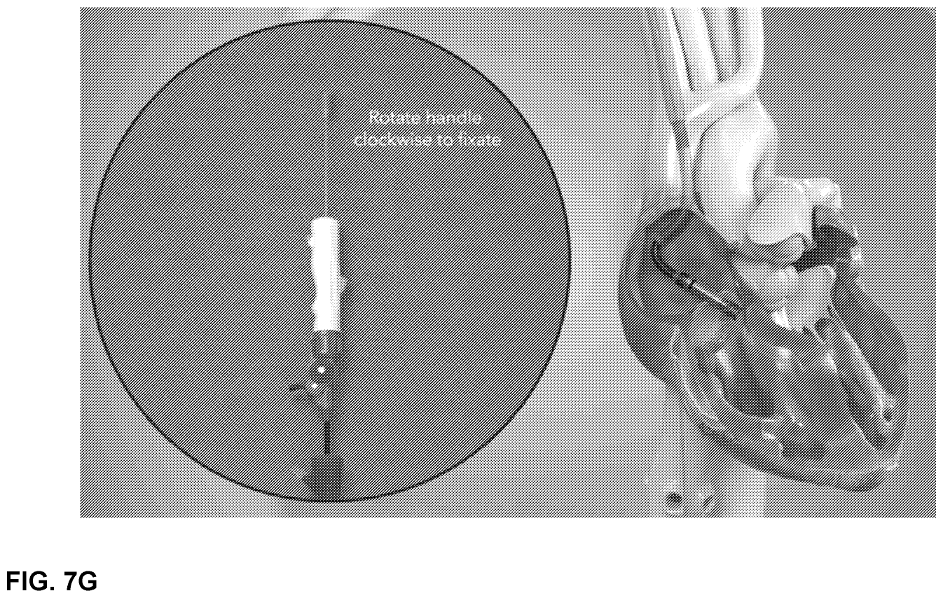

FIGS. 7A-7G are conceptual diagrams of an example technique for operating a delivery device (e.g., delivery device 40) in accordance with techniques of this disclosure, to deliver a medical device (e.g., IMD 100) to an implant location (e.g. TOK) within the heart of a patient. The heart shown in FIGS. 7A-7G has a relatively small right atrium. Accordingly, the usage of delivery device 40 as discussed in FIG. 4C is described in greater detail here. As shown in FIGS. 7A-7G, the medical device (e.g., IMD 100), such as a leadless cardiac rhythm management device, may include a distal fixation helix. The fixation helix may include a first electrode (e.g. first electrode 112), and the medical device may further include a second electrode (e.g., second electrode 114) configured to contact atrial wall tissue without penetration thereof.

In FIG. 7A, ultrasound is used to visualize the internal jugular vein. The internal jugular vein is a major vein located in the neck commonly used as an access point for the insertion of IMDs, such as IMD 100. The internal jugular vein, which has a relatively large diameter, provides a direct route to the superior vena cava, which may be useful for various medical procedures including the implant of IMD 100.

After identifying an access point 700 into the internal jugular vein, a clinician may insert a guidewire 702 through access point 700 and navigate guidewire 702 proximate a target implant site within the heart (or other location). Guidewire 702 may be a thin, flexible wire configured to guide the placement of other devices (e.g., an introducer, delivery device 40, etc.), allowing a clinician to navigate the other devices through blood vessels, organs, or other anatomical structures with precision and safety. In some examples, guidewire 702 may be radiopaque, such that guidewire 702 can be seen on X-ray or fluoroscopy images. In this way, a clinician may track the position of the guidewire in real-time during procedures.

In FIG. 7B, a clinician may advance an introducer 704 with a dilator (not shown) received therein, over guidewire 702. The clinician may navigate the introducer 704 with dilator therein to a location (e.g. the superior vena cava) proximate the target implant site. Introducer 704 may be configured to facilitate the placement of delivery device 40 and/or other medical instruments into the body, e.g. to the right atrium and/or right ventricle. The introducer may provide a safe and controlled means of accessing the vascular system or other body cavities, while preventing or impeding flow of blood out of the vascular system.

The clinician may remove guidewire 702 and the dilator. As shown in FIG. 7C, a clinician may insert delivery device 40 into introducer 704, and advance delivery device 400 until distal receptacle 54 thereof (with IMD 100 positioned therein) is in the right atrium. In some examples, a clinician may place a secondary marker 706 to help cue (e.g., indicate the orientation of) the fluoroscopic view and provide a reference point for the movement of delivery device 40. Secondary marker 706, which can comprise a catheter such as a diagnostic catheter, may be placed in the coronary sinus and act as a landmark for navigation because the target implant site (e.g., TOK) may be between the coronary sinus and tricuspid valve. In FIG. 7C, the secondary marker 706 may be advanced through a femoral access point, but any access point may be used.

Using delivery device 40, a clinician may position distal receptacle 54 containing IMD 100 at a location proximate the target implant site. The location of the body may include an atrium of a heart. The location or target implant site may be the Triangle of Koch (TOK). As shown in FIG. 7D, a clinician may use an imaging modality, such as fluoroscopy, to view a position of distal receptacle 54 and/or IMD 100 within the body of the patient. As described above (e.g., FIGS. 4A-4C), the clinician may navigate and orient distal receptacle 54 by controlling various mechanisms of delivery device 40. For example, the clinician may actuate inner shaft deflection mechanism 44 to deflect inner shaft 46 (e. g, deflect a distal end portion of inner shaft 46 laterally) and navigate at least a portion of distal receptacle 54 into the right ventricle, across the tricuspid valve and oriented toward the septum.

Following this step, the clinician may also rotate delivery device 40 (e.g., rotating first and second handle portions 42, 48 together) relative to introducer 704 and the heart, generally about the axis of introducer 704, in such a manner as to cause distal receptacle 54 and/or IMD 100 to rotate from a more anteriorly-pointed orientation to a less anteriorly-pointed orientation (e.g., rotating the distal tip of distal receptacle 54 toward the patient's left arm and/or aligning the axis of the distal receptacle 54 more closely with the coronal plane), while distal receptacle 54 remains across the tricuspid valve and oriented toward the septum. The clinician may then withdraw delivery device 40 in the proximal direction relative to introducer 40 and the heart, which moves distal receptacle 54 toward or completely into the right atrium; such a maneuver leaves a smaller distal portion of the distal receptacle 54 (or none of the distal receptacle 54) on the ventricular side of the tricuspid valve. Additionally, as a result of the proximal withdrawal, a distal portion of inner and/or outer shafts 46, 52 may move to the right within the right atrium and/or superior vena cava, and abut the right wall of the right atrium and/or superior vena cava.

Next, the clinician may deflect outer shaft 52 and thereby attain the configuration shown in FIGS. 7D, 7F and/or 7G. The clinician may accomplish this configuration by actuating outer shaft deflection mechanism 50 to deflect outer shaft 52. The clinician may also rotate second handle portion 48 relative to first handle portion 42 (e.g., about 180 degrees relative to first handle portion 42) and thereby rotate outer shaft 52 relative to inner shaft 46. As a result of the deflection/curving of outer shaft 52, distal receptacle 54 may withdraw from the tricuspid valve (if distal receptacle 54 is not already completely within the right atrium). The clinician may now position distal receptacle 54 at the target implant site in the right atrium, or confirm that distal receptacle 54 is already so positioned as a result of reaching the position and orientation shown in 7D, 7F and/or 7G.

To aid in properly positioning distal receptacle 54 or confirming its proper positioning or orientation, the clinician may use one or more techniques. For example, throughout the procedure, the clinician may rely on fluoroscopic images to visualize internal structures and observe or track the position of delivery device 40 (e.g., in relation to secondary marker 706, which may be proximate the target implant site). This may involve observing via fluoroscopy or otherwise that the axis of distal receptacle 54 is pointed at or toward the left ventricular apex, as may be seen in FIG. 7G. In some examples, and as shown in FIG. 7E, the clinician may introduce a contrast agent 708 (e.g., via distal receptacle 54 and proximate the implant site), such as an iodine-based contrast, to visualize blood vessels, organs, etc. The clinician may analyze the flow of contrast within the heart to determine the location of distal receptacle 54 relative to landmark structures near the target implant site. For example, the clinician may observe the flow of contrast to determine the location of the tricuspid valve and the coronary sinus (e.g., because of the contrast agent periodically flowing into the tricuspid valve and the coronary sinus). The target implant site or TOK may be the tissue between the tricuspid valve and the coronary sinus, and the clinician may observe unique contrast agent cloud movement in that area.

Additionally or alternatively, the clinician may use an electrogram to identify the position of distal receptacle 54. For example, an electrode on distal receptacle 54 may contact tissue of the target implant site and obtain an electrogram. The electrogram may indicate various waveforms, such as a set (e.g., one or more) of P-waves and a set of R-waves. A P-wave may be the first wave of the electrogram and represent the electrical depolarization (contraction) of the atria. An R-wave may follow the P-wave and represent the rapid electrical depolarization of the ventricles, which triggers the main pumping action of the heart.

Depending on the proximity of distal receptacle 54 to the left atrium or the right ventricle, the amplitudes of the P-waves and R-waves may differ. For example, if distal receptacle 54 is closer to the left atrium, the amplitude of the P-waves may be greater and the amplitude of the R-waves may be smaller. Conversely, if distal receptacle 54 is closer to the LV, the amplitude of the R-waves may be greater and the amplitude of the P-waves may be smaller. Accordingly, when distal receptacle 54 is positioned at the target implant site, the ratio of the amplitude of the P-waves and R-waves may be between a range. In other words, the ratio of the P-waves and R-waves being greater than a lower threshold value and less than an upper threshold value may indicate that distal receptacle 54 is properly positioned at the target implant site. In some examples, the clinician may use other aspects of the electrogram (e.g., timing, morphology, etc.) to determine the position of distal receptacle 54.

In addition to positioning distal receptacle 54 at the target implant site, the clinician may need to properly orient distal receptacle 54 with respect to the target implant site. Using an appropriate imaging modality (e.g., fluoroscopy), the clinician may view a first image of IMD 100 including first electrode 112 (e.g., the image on the right-hand side of FIG. 7D). Based on the appearance of first electrode 112 (e.g., the orthogonal projection of first electrode 112) in the first image (e.g., resembling or forming one or more closed loops), the clinician may alter an orientation of IMD 100. In some examples, altering the orientation of IMD 100 may include rotating delivery device 40 and/or changing the shaft deflections, or rotating second handle portion 48 relative to first handle portion 42. After altering the orientation of IMD 100, the clinician may fixate IMD 100 to tissue of the patient. In some examples, the clinician may fixate IMD 100 to tissue of the patient while IMD 100 is in the altered orientation.

In some examples, when altering the orientation of the medical device (IMD 100), the clinician may view a second image of IMD 100 (e.g., the image on the right-hand side of FIG. 7F). Based on the appearance of first electrode 112 in the second image, the clinician may recognize that IMD 100 is in a proper orientation for subsequent fixation. For example, as shown in FIG. 7F, the appearance of first electrode 112 in the second image may resemble a sinusoidal wave (e.g., which may indicate the proper orientation for fixation). The appearance of first electrode 112 in the first image may resemble something other than a sinusoidal wave. For example, the appearance of first electrode 112 in the first image may resemble at least one closed loop.

Based on one or more of the above criteria, the clinician may confirm that the position and orientation of IMD 100 is proper for implantation. Consequently, as shown in FIG. 7G, the clinician may fixate IMD 100 (e.g., by rotating tether assembly 66 until the clinician receives torsional feedback). When IMD 100 is secured, first electrode 112 may penetrate through the wall tissue of a first chamber (e.g., the RA) into wall tissue of a second chamber (e.g., ventricular myocardium 108 of the LV), and second electrode 114 may be configured to contact atrial wall tissue without penetration thereof.

The following examples are illustrative of the techniques described herein.

Example 1: A delivery device includes a first handle portion including an inner shaft deflection mechanism; an inner shaft coupled to the first handle portion, wherein the inner shaft defines a longitudinally extending lumen, wherein the inner shaft mechanically supports a distal receptacle configured to receive an implantable medical device, and wherein the inner shaft is configured to deflect in a first deflection plane in response to actuation of the inner shaft deflection mechanism; a second handle portion including an outer shaft deflection mechanism, wherein the second handle portion is configured to rotate relative to the first handle portion; and an outer shaft coupled to the second handle portion, wherein the outer shaft surrounds at least a portion of the inner shaft, wherein the outer shaft is configured to deflect in a second deflection plane in response to actuation of the outer shaft deflection mechanism, and wherein rotation of the second handle portion relative to the first handle portion causes the second deflection plane to rotate relative to the first deflection plane.

Example 2: The delivery device of example 1, wherein a distal end of the inner shaft is proximal to the distal receptacle, and wherein an inner diameter of the outer shaft is smaller than an outer diameter of the distal receptacle.

Example 3: The delivery device of example 1 or 2, further including a deflection locking mechanism configured to maintain an outer shaft configuration when the deflection locking mechanism is actuated.

Example 4: The delivery device of any of examples 1 to 3, further including a rotation locking mechanism configured to maintain a rotational angle of the second handle portion relative to the first handle portion.

Example 5: The delivery device of any of examples 1 to 4, wherein a first pull wire extends from the inner shaft deflection mechanism to the inner shaft, and wherein a second pull wire extends from the outer shaft deflection mechanism to the outer shaft.

Example 6: The delivery device of any of examples 1 to 5, wherein the longitudinally extending lumen is sized to receive a tether assembly.

Example 7: The delivery device of any of examples 1 to 6, wherein an inner diameter of a distal tip portion of the outer shaft and an outer diameter of the inner shaft are sized to prevent fluid from flowing between the outer shaft and the inner shaft at the distal tip portion of the outer shaft.

Example 8: The delivery device of any of examples 1 to 7, further including visual indicia that indicate a degree of rotation of the second handle portion relative to the first handle portion.

Example 9: The delivery device of any of examples 1 to 8, wherein the outer shaft deflection mechanism includes a lever, and wherein deflection of the outer shaft causes corresponding deflection of the inner shaft.

Example 10: The delivery device of any of examples 1 to 9, wherein the inner shaft deflection mechanism is configured to cause the inner shaft to deflect in the first plane when the inner shaft deflection mechanism is actuated, and wherein the outer shaft deflection mechanism is configured to cause the outer shaft to deflect in the second plane when the inner shaft deflection mechanism is actuated.

Example 11: The delivery device of any of examples 1 to 10, wherein the second handle portion is configured to slide along a longitudinal axis of the delivery device relative to the first handle portion.

Example 12: The delivery device of any of examples 1 to 11, wherein the first handle portion and the second handle portion are coupled together in an integral handle unit.

Example 13: The delivery device of any of examples 1 to 12, wherein the first handle portion is disposed at a first longitudinal position on a longitudinal axis of the delivery device, wherein the second handle portion is disposed at a second longitudinal position on the longitudinal axis of the delivery device, and wherein the first longitudinal position is different from the second longitudinal position.

Example 14: The delivery device of example 13, wherein the second longitudinal position is distal to the first longitudinal position.

Example 15: The delivery device of any of examples 1 to 14, wherein the distal receptacle defines a cup.

Example 16: The delivery device of any of examples 1 to 15, wherein deflection of a portion of the outer shaft causes deflection of an underlying portion of the inner shaft.

Example 17: A system includes an implantable medical device; and a delivery device includes a first handle portion including an inner shaft deflection mechanism; an inner shaft coupled to the first handle portion, wherein the inner shaft defines a longitudinally extending lumen, wherein the inner shaft mechanically supports a distal receptacle configured to receive an implantable medical device, and wherein the inner shaft is configured to deflect in a first deflection plane in response to actuation of the inner shaft deflection mechanism; a second handle portion including an outer shaft deflection mechanism, wherein the second handle portion is configured to rotate relative to the first handle portion; and an outer shaft coupled to the second handle portion, wherein the outer shaft surrounds at least a portion of the inner shaft; wherein the outer shaft is configured to deflect in a second deflection plane in response to actuation of the outer shaft deflection mechanism, and wherein rotation of the second handle portion relative to the first handle portion causes the second deflection plane to rotate relative to the first deflection plane.

Example 18: The system of example 17, wherein the implantable medical device includes: an elongated housing extending from a proximal end to a distal end, the elongated housing being configured to be implanted wholly within a chamber of a heart; a first electrode extending distally from the distal end of the elongated housing, the first electrode includes an elongated body defining a helix; and a second electrode disposed at or near a distal end of the elongated housing, wherein the second electrode is configured to contact wall tissue of the chamber without penetration of the wall tissue of the chamber by the second electrode.

Example 19: The system of example 17 or 18, wherein a distal end of the inner shaft is proximal to the distal receptacle, and wherein an inner diameter of the outer shaft is smaller than an outer diameter of the distal receptacle.

Example 20: The system of any of examples 17 to 19, further including a deflection locking mechanism configured to maintain an outer shaft configuration when the deflection locking mechanism is actuated.

Example 21: The system of any of examples 17 to 20, further including a rotation locking mechanism configured to maintain a rotational angle of the second handle portion relative to the first handle portion.

Example 22: The system of any of examples 17 to 21, wherein a first pull wire extends from the inner shaft deflection mechanism to the inner shaft, and wherein a second pull wire extends from the outer shaft deflection mechanism to the outer shaft.

Example 23: The system of any of examples 17 to 22, wherein the longitudinally extending lumen is sized to receive a tether assembly.

Example 24: The system of any of examples 17 to 23, wherein an inner diameter of the outer shaft and an outer diameter of the inner shaft are sized to prevent fluid from flowing between the outer shaft and the inner shaft.

Example 25: The system of any of examples 17 to 24, wherein the implantable medical device further includes a ramp extending distally from the distal end of the elongated housing, wherein the second electrode is disposed on the ramp, and wherein the ramp is configured to: promote contact between the second electrode and wall tissue of the chamber without penetration of the wall tissue of the chamber by the second electrode; and separate the second electrode from the distal end of the elongated housing by a fixed distance.

Example 26: A method includes inserting a distal receptacle of a delivery device into an access site on a body of a patient, wherein the distal receptacle is carrying an implantable medical device; navigating the delivery device toward an implant site; one or more of: actuating an inner shaft deflection mechanism of the delivery device to cause an inner shaft of the delivery device to deflect in a first deflection plane, actuating an outer shaft deflection mechanism of the delivery device to cause an outer shaft of the delivery device to deflect in a second deflection plane, or rotating a second handle portion of the delivery device relative to a first handle portion of the delivery device, wherein an angle between the first deflection plane and the second deflection plane is based on an amount of rotation of the second handle portion relative to the first handle portion; and securing the implantable medical device to the implant site.

Example 27: The method of example 26, wherein the implant site includes the Triangle of Koch.

Example 28: The method of any of examples 26 or 27, wherein the implantable medical device includes a leadless cardiac rhythm management device.

Example 29: The method of any of examples 26 to 28, wherein a fixation helix of the implantable medical device includes a first electrode, and wherein the implantable medical device further includes a second electrode configured to contact atrial wall tissue without penetration thereof.

Example 30: The method of any of examples 26 to 29, wherein altering the orientation of the medical device includes rotating the delivery system.

Example 31: A method of delivering a medical device having a distal fixation helix includes positioning the medical device in a location in a body of a patient using an associated delivery device; viewing a first image of the positioned medical device including the distal fixation helix; based on the appearance of the distal fixation helix in the first image, altering an orientation of the medical device; after altering the orientation of the medical device, fixating the medical device to tissue of the patient.

Example 32: The method of example 31, wherein fixating the medical device to tissue of the patient includes fixating the medical device while in the altered orientation.

Example 33: The method of example 31 or 32, further including, after altering the orientation of the medical device, viewing a second image of the medical device, and, based on the appearance of the distal fixation helix in the second image, recognizing that the medical device is in a proper orientation for subsequent fixation.

Example 34: The method of example 33, wherein the appearance of the distal fixation helix in the second image resembles a sinusoidal wave.

Example 35: The method of any of examples 31 to 34, wherein the appearance of the distal fixation helix in the first image resembles other than a sinusoidal wave.

Example 36: The method of example 35, wherein the appearance of the distal fixation helix in the first image resembles at least one closed loop.

Example 37: The method of any of examples 31 to 36, wherein the location in the body of the patient includes an atrium of a heart.

Example 38: The method of example 37, wherein the location includes the Triangle of Koch.

Example 39: The method of any of examples 31 to 38, wherein the medical device includes a leadless cardiac rhythm management device.

Example 40: The method of example 39, wherein the fixation helix includes a first electrode, and the medical device further includes a second electrode configured to contact atrial wall tissue without penetration thereof.

Example 41: The method of any of examples 31 to 40, wherein altering the orientation of the medical device includes rotating the delivery system.

Example 42: A method of delivering a medical device using a delivery device having an inner shaft, an outer shaft surrounding the inner shaft, and a distal receptacle coupled to the inner shaft and containing the medical device includes advancing the shafts so as to position the receptacle in the right atrium; while the receptacle is in the right atrium, operating the delivery device so as to bend a distal portion of the inner shaft from a straight or less bent configuration to a more bent configuration; causing at least a distal tip of the receptable to pass the tricuspid valve toward the right ventricle; while the distal portion of the inner shaft is in the more bent configuration, retracting the receptacle toward or fully into the right atrium; after retracting the receptacle, operating the delivery device so as to bend a distal portion of the outer shaft from a straight or less bent configuration to a more bent configuration, thereby moving a distal tip of the receptacle toward the Triangle of Koch.

Example 43: The method of example 42, further including rotating the inner shaft while the distal portion of the inner shaft is in the more bent configuration, and before retracting the receptacle.

Example 44: The method of example 42 or example 43, further including using the delivery system to fixate the medical device at the Triangle of Koch.

Example 45: The method of example 44, wherein fixating the medical device includes rotating it so as to screw a fixation helix of the medical device into tissue.

Example 46: The method of any of examples 42 to 45, further including viewing, on an image, an orientation of the medical device after moving the distal tip of the receptacle toward the Triangle of Koch.

Example 47: The method of example 46, further including adjusting the orientation of the medical device based on the orientation viewed in the image.

Example 48: The method of example 47, wherein adjusting the orientation of the medical device includes rotating the delivery device.

Example 49: The method of any of examples 42 to 48, wherein the orientation of the medical device is determined by viewing the shape made by the fixation helix of the medical device in the image.

Example 50: A delivery device includes a first handle portion including an inner shaft deflection mechanism; an inner shaft coupled to the first handle portion, wherein the inner shaft defines a longitudinally extending lumen, wherein the inner shaft mechanically supports a distal receptacle configured to receive an implantable medical device, and wherein the inner shaft is configured to deflect in a first deflection plane in response to actuation of the inner shaft deflection mechanism; a second handle portion including an outer shaft deflection mechanism; and an outer shaft coupled to the second handle portion, wherein the outer shaft surrounds at least a portion of the inner shaft; wherein the outer shaft is configured to deflect in a second deflection plane, distinct from the first deflection plane, in response to actuation of the outer shaft deflection mechanism; wherein a distal end of the inner shaft is proximal to the distal receptacle, and wherein an inner diameter of the outer shaft is smaller than an outer diameter of the distal receptacle.

Example 51: The delivery device of example 50, wherein the second handle portion is configured to rotate relative to the first handle portion, and wherein rotation of the second handle portion relative to the first handle portion causes the second deflection plane to rotate relative to the first deflection plane.

Example 52: The delivery device of example 50 or 51, further including a deflection locking mechanism configured to maintain an outer shaft configuration when the deflection locking mechanism is actuated.

Example 53: The delivery device of any of examples 50 to 52, further including a rotation locking mechanism configured to maintain a rotational angle of the second handle portion relative to the first handle portion.

Example 54: The delivery device of any of examples 50 to 53, wherein a first pull wire extends from the inner shaft deflection mechanism to the inner shaft, and wherein a second pull wire extends from the outer shaft deflection mechanism to the outer shaft.

Example 55: The delivery device of any of examples 50 to 54, wherein the longitudinally extending lumen is sized to receive a tether assembly.

Example 56: The delivery device of any of examples 50 to 55, wherein an inner diameter of a distal tip portion of the outer shaft and an outer diameter of the inner shaft are sized to prevent fluid from flowing between the outer shaft and the inner shaft at the distal tip portion of the outer shaft.

Example 57: The delivery device of any of examples 50 to 56, further including visual indicia that indicate a degree of rotation of the second handle portion relative to the first handle portion.

Example 58: The delivery device of any of examples 50 to 57, wherein the outer shaft deflection mechanism includes a lever, and wherein deflection of the outer shaft causes corresponding deflection of the inner shaft.

Example 59: The delivery device of any of examples 50 to 58, wherein the inner shaft deflection mechanism is configured to cause the inner shaft to deflect in the first plane when the inner shaft deflection mechanism is actuated, and wherein the outer shaft deflection mechanism is configured to cause the outer shaft to deflect in the second plane when the inner shaft deflection mechanism is actuated.

Example 60: The delivery device of any of examples 50 to 59, wherein the second handle portion is configured to slide along a longitudinal axis of the delivery device relative to the first handle portion.

Example 61: The delivery device of any of examples 50 to 60, wherein the first handle portion and the second handle portion are coupled together in an integral handle unit.

Example 62: A delivery device includes an inner shaft, wherein the inner shaft defines a longitudinally extending lumen, and wherein the inner shaft mechanically supports a distal receptacle configured to receive an implantable medical device; and an outer shaft, wherein the outer shaft surrounds at least a portion of the inner shaft, wherein a distal end of the inner shaft is proximal to the distal receptacle, wherein an inner diameter of the outer shaft is smaller than an outer diameter of the distal receptacle, and wherein a distal portion of the inner shaft and a distal portion of the outer shaft are each deflectable remotely, and independently of the other.

Example 63: The delivery device of example 62, further includes a first handle portion including an inner shaft deflection mechanism, wherein the inner shaft is coupled to the first handle portion, and wherein the inner shaft is configured to deflect in a first deflection plane in response to actuation of the inner shaft deflection mechanism; and a second handle portion including an outer shaft deflection mechanism, wherein the outer shaft is coupled to the second handle portion, and wherein the outer shaft is configured to deflect in a second deflection plane in response to actuation of the outer shaft deflection mechanism.

Example 64: The delivery device of example 63, wherein the second handle portion is configured to rotate relative to the first handle portion, and wherein rotation of the second handle portion relative to the first handle portion causes the second deflection plane to rotate relative to the first deflection plane.

Example 65: The delivery device of any of examples 62 to 64, further including a deflection locking mechanism configured to maintain an outer shaft configuration when the deflection locking mechanism is actuated.

Example 66: The delivery device of any of examples 62 to 65, further including a rotation locking mechanism configured to maintain a rotational angle of the second handle portion relative to the first handle portion.

Example 67: The delivery device of any of examples 62 to 66, wherein a first pull wire extends from the inner shaft deflection mechanism to the inner shaft, and wherein a second pull wire extends from the outer shaft deflection mechanism to the outer shaft.

Example 68: The delivery device of any of examples 62 to 67, wherein the longitudinally extending lumen is sized to receive a tether assembly.

Example 69: The delivery device of any of examples 62 to 68, wherein an inner diameter of a distal tip portion of the outer shaft and an outer diameter of the inner shaft are sized to prevent fluid from flowing between the outer shaft and the inner shaft at the distal tip portion of the outer shaft.

Example 70: The delivery device of any of examples 62 to 69, further including visual indicia that indicate a degree of rotation of the second handle portion relative to the first handle portion.

Example 71: The delivery device of any of examples 62 to 70, wherein the outer shaft deflection mechanism includes a lever, and wherein deflection of the outer shaft causes corresponding deflection of the inner shaft.

Example 72: The delivery device of any of examples 62 to 71, wherein the inner shaft deflection mechanism is configured to cause the inner shaft to deflect in the first plane when the inner shaft deflection mechanism is actuated, and wherein the outer shaft deflection mechanism is configured to cause the outer shaft to deflect in the second plane when the inner shaft deflection mechanism is actuated.

Example 73: The delivery device of any of examples 62 to 72, wherein the second handle portion is configured to slide along a longitudinal axis of the delivery device relative to the first handle portion.

Example 74: The delivery device of any of examples 62 to 73, wherein the first handle portion and the second handle portion are coupled together in an integral handle unit.

Example 75: The delivery device of any of examples 62 to 74, further including a normal-atrium deflected configuration in which the inner shaft is deflected laterally from its longitudinal axis by about 90 degrees, and the outer shaft is not independently deflected.

Example 76: The delivery device of any of examples 62 to 75, further including a normal-atrium deflected configuration in which the distal receptacle is oriented about 90 degrees laterally from the longitudinal axis of the inner shaft, and a distal tip of the distal receptacle is spaced laterally from the longitudinal axis of the inner shaft by a distance greater than the length of the receptacle but less than twice the length of the receptacle.

Example 77: The delivery device of any of examples 62 to 76, further including a large-atrium deflected configuration in which the outer shaft is deflected laterally from its longitudinal axis by about 90 degrees, and the inner shaft is not independently deflected.

Example 78: The delivery device of any of examples 62 to 77, further including a large-atrium deflected configuration in which the distal receptacle is oriented about 90 degrees laterally from the longitudinal axis of the inner shaft, and a distal tip of the distal receptacle is spaced laterally from the longitudinal axis of the inner shaft by a distance more than twice the length of the receptacle.

Example 79: The delivery device of any of examples 62 to 78, further including a small-atrium deflected configuration in which the outer shaft is deflected laterally from its longitudinal axis in a first lateral direction, and the inner shaft is deflected laterally in a second lateral direction opposite the first lateral direction.

Example 80: The delivery device of any of examples 62 to 79, further including a small-atrium deflected configuration in which the distal receptacle is oriented about 90 degrees laterally from the longitudinal axis of the inner shaft, and a distal tip of the distal receptacle is spaced laterally from the longitudinal axis of the inner shaft by a distance less than the length of the receptacle.

Example 81: A method includes inserting a distal receptacle of a delivery device into an access site on a body of a patient, wherein the distal receptacle is carrying an implantable medical device; navigating the delivery device toward an implant site using an imaging modality; actuating an inner shaft deflection mechanism of the delivery device to cause an inner shaft of the delivery device to deflect in a first deflection plane; rotating a second handle portion of the delivery device relative to a first handle portion of the delivery device; actuating an outer shaft deflection mechanism of the delivery device to cause an outer shaft of the delivery device to deflect in a second deflection plane, wherein an angle between the first deflection plane and the second deflection plane is based on an amount of rotation of the second handle portion relative to the first handle portion; and securing the implantable medical device to the implant site.

Example 82: The method of example 81, wherein the imaging modality is fluoroscopy.

Example 83: The method of example 81 or 82, further includes contacting tissue at the implant site; obtaining an electrogram from the tissue; and determining that the distal receptacle is properly positioned at the implant site based on a set of P-waves and a set of R-waves from the electrogram.

Example 84: The method of example 83, wherein determining that the distal receptacle is properly positioned based on the set of P-waves and the set of R-waves includes: determining a ratio between at least one P-wave of the set of P-waves and at least one R-wave of the set of R-waves; and determining whether the ratio is equal to or greater than a lower threshold and equal to or less than an upper threshold.

Example 85: The method of any of examples 81 to 84, further includes introducing a contrast agent proximate the implant site; and determining that the distal receptacle is properly positioned based on a flow of the contrast agent.

Example 86: The method of any of examples 81 to 85, wherein securing the implantable medical device to the implant site includes using a tether assembly.

Example 87: A delivery device for implantation of a leadless cardiac stimulation device in an atrium of a patient, the delivery device includes at least one shaft extending along a longitudinal axis; wherein the at least one shaft supports a distal receptacle that receives the leadless cardiac stimulation device; wherein the at least one shaft is deflectable to: a normal-atrium deflected configuration in which the distal receptacle is oriented about 90 degrees laterally from the longitudinal axis of the at least one shaft, and a distal tip of the distal receptacle is spaced laterally from the longitudinal axis of the at least one shaft by a distance greater than the length of the receptacle but less than twice the length of the receptacle; a large-atrium deflected configuration in which the distal receptacle is oriented about 90 degrees laterally from the longitudinal axis of the at least one shaft, and a distal tip of the distal receptacle is spaced laterally from the longitudinal axis of the at least one shaft by a distance more than twice the length of the receptacle; and a small-atrium deflected configuration in which the distal receptacle is oriented about 90 degrees laterally from the longitudinal axis of the at least one shaft, and a distal tip of the distal receptacle is spaced laterally from the longitudinal axis of the at least one shaft by a distance less than the length of the receptacle.

Example 88: The delivery system of example 87, wherein the at least one shaft includes: an inner shaft, wherein the inner shaft defines a longitudinally extending lumen, and an outer shaft, wherein the outer shaft surrounds at least a portion of the inner shaft, wherein a distal portion of the inner shaft and a distal portion of the outer shaft are each deflectable remotely, and independently of the other.

Example 89: The delivery system of example 88, wherein the inner shaft extends distally beyond a distal end of the outer shaft, and the inner shaft mechanically supports the distal receptacle.

Example 90: The delivery system of example 88, wherein the outer shaft is rotatable around the inner shaft.

Example 91: The delivery system of example 88, wherein, in the normal-atrium deflected configuration, the inner shaft is deflected laterally from its longitudinal axis by about 90 degrees, and the outer shaft is not independently deflected.

Example 92: The delivery system of example 88 or example 91, wherein, in the large-atrium deflected configuration, the outer shaft is deflected laterally from its longitudinal axis by about 90 degrees, and the inner shaft is not independently deflected.

Example 93: The delivery system of example 88, 91 or 92, wherein, in the small-atrium deflected configuration, the outer shaft is deflected laterally from its longitudinal axis in a first lateral direction, and the inner shaft is deflected laterally in a second lateral direction opposite the first lateral direction.

Example 94: The delivery system of example 87, wherein the distal receptacle has a larger outer diameter than an outer diameter of the at least one shaft.

Various examples of the disclosure have been described. Any combination of the described systems, operations, or functions is contemplated. These and other examples are within the scope of the following claims.

1-15. (canceled)

16: A delivery device comprising:
an inner shaft,
wherein the inner shaft defines a longitudinally extending lumen, and
wherein the inner shaft mechanically supports a distal receptacle configured to receive an implantable medical device; and
an outer shaft,
wherein the outer shaft surrounds at least a portion of the inner shaft,
wherein a distal end of the inner shaft is proximal to the distal receptacle,
wherein an inner diameter of the outer shaft is smaller than an outer diameter of the distal receptacle, and
wherein a distal portion of the inner shaft and a distal portion of the outer shaft are each deflectable remotely, and independently of the other.

17: The delivery device of claim 16, further comprising:
a first handle portion comprising an inner shaft deflection mechanism, wherein the inner shaft is coupled to the first handle portion, and wherein the inner shaft is configured to deflect in a first deflection plane in response to actuation of the inner shaft deflection mechanism; and
a second handle portion comprising an outer shaft deflection mechanism, wherein the outer shaft is coupled to the second handle portion, and wherein the outer shaft is configured to deflect in a second deflection plane in response to actuation of the outer shaft deflection mechanism.

18: The delivery device of claim 17, wherein the second handle portion is configured to rotate relative to the first handle portion, and wherein rotation of the second handle portion relative to the first handle portion causes the second deflection plane to rotate relative to the first deflection plane.

19: The delivery device of claim 16, further comprising a deflection locking mechanism configured to maintain an outer shaft configuration when the deflection locking mechanism is actuated.

20: The delivery device of claim 16, further comprising a rotation locking mechanism configured to maintain a rotational angle of the second handle portion relative to the first handle portion.

21: The delivery device of claim 16, wherein a first pull wire extends from the inner shaft deflection mechanism to the inner shaft, and wherein a second pull wire extends from the outer shaft deflection mechanism to the outer shaft.

22: The delivery device of claim 16, wherein the longitudinally extending lumen is sized to receive a tether assembly.

23: The delivery device of claim 16, wherein an inner diameter of a distal tip portion of the outer shaft and an outer diameter of the inner shaft are sized to prevent fluid from flowing between the outer shaft and the inner shaft at the distal tip portion of the outer shaft.

24: The delivery device of claim 16, further comprising visual indicia that indicate a degree of rotation of the second handle portion relative to the first handle portion.

25: The delivery device of claim 16, wherein the outer shaft deflection mechanism comprises a lever, and wherein deflection of the outer shaft causes corresponding deflection of the inner shaft.

26: The delivery device of claim 16, wherein the inner shaft deflection mechanism is configured to cause the inner shaft to deflect in the first plane when the inner shaft deflection mechanism is actuated, and wherein the outer shaft deflection mechanism is configured to cause the outer shaft to deflect in the second plane when the inner shaft deflection mechanism is actuated.

27: The delivery device of claim 16, wherein the second handle portion is configured to slide along a longitudinal axis of the delivery device relative to the first handle portion.

28: The delivery device of claim 16, wherein the first handle portion and the second handle portion are coupled together in an integral handle unit.

29: A delivery device for implantation of a leadless cardiac stimulation device in an atrium of a patient, the delivery device comprising:
at least one shaft extending along a longitudinal axis; wherein the at least one shaft supports a distal receptacle that receives the leadless cardiac stimulation device;
wherein the at least one shaft is deflectable to:
(a) a normal-atrium deflected configuration in which the distal receptacle is oriented about 90 degrees laterally from the longitudinal axis of the at least one shaft, and a distal tip of the distal receptacle is spaced laterally from the longitudinal axis of the at least one shaft by a distance greater than the length of the receptacle but less than twice the length of the receptacle;
(b) a large-atrium deflected configuration in which the distal receptacle is oriented about 90 degrees laterally from the longitudinal axis of the at least one shaft, and a distal tip of the distal receptacle is spaced laterally from the longitudinal axis of the at least one shaft by a distance more than twice the length of the receptacle; and
(c) a small-atrium deflected configuration in which the distal receptacle is oriented about 90 degrees laterally from the longitudinal axis of the at least one shaft, and a distal tip of the distal receptacle is spaced laterally from the longitudinal axis of the at least one shaft by a distance less than the length of the receptacle.

30: The delivery system of claim 29, wherein the at least one shaft comprises:
an inner shaft,
wherein the inner shaft defines a longitudinally extending lumen, and
an outer shaft,
wherein the outer shaft surrounds at least a portion of the inner shaft,
wherein a distal portion of the inner shaft and a distal portion of the outer shaft are each deflectable remotely, and independently of the other.

31: The delivery system of claim 30, wherein the inner shaft extends distally beyond a distal end of the outer shaft, and the inner shaft mechanically supports the distal receptacle.

32: The delivery system of claim 30, wherein the outer shaft is rotatable around the inner shaft.

33: The delivery system of claim 30, wherein, in the normal-atrium deflected configuration, the inner shaft is deflected laterally from its longitudinal axis by about 90 degrees, and the outer shaft is not independently deflected.

34: The delivery system of claim 30, wherein, in the large-atrium deflected configuration, the outer shaft is deflected laterally from its longitudinal axis by about 90 degrees, and the inner shaft is not independently deflected.

35: The delivery system of claim 30, wherein, in the small-atrium deflected configuration, the outer shaft is deflected laterally from its longitudinal axis in a first lateral direction, and the inner shaft is deflected laterally in a second lateral direction opposite the first lateral direction.
